(19) United States
(12) Patent Application Publication
(10) Pub. No.: US 2023/0183665 A1
(43) Pub. Date: Jun. 15, 2023

(54) THERMOSTABLE GLUCOCEREBROSIDASE

(71) Applicant: TEIKYO UNIVERSITY, Tokyo (JP)

(72) Inventors: Jinichiro KOGA, Utsunomiya (JP); Hisakazu YAMANE, Utsunomiya (JP); Koji MIYAMOTO, Utsunomiya (JP); Makoto YAZAWA, Utsunomiya (JP); Tomoyoshi KUBOTA, Utsunomiya (JP)

(21) Appl. No.: 17/924,704

(22) PCT Filed: Feb. 10, 2021
(86) PCT No.: PCT/JP2021/004999

§ 371 Date: Nov. 11, 2022

(30) Foreign Application Priority Data
May 21, 2020 (JP) 2020-088950

(51) Int. Cl. C12N 9/24; C12N 15/70; C12P 13/02; A23L 33/185; A23L 33/115; A23L 33/00

(52) U.S. Cl. CPC C12N 9/2402; C12Y 302/01045; C12N 15/70; C12P 13/02; A23L 33/185; A23L 33/115; A23L 33/40; A61K 38/00

(57) ABSTRACT

Provided is a protein having glucocerebrosidase activity and further having thermostability. The object is attained by a protein which is derived from a plant, belongs to glycoside hydrolase family 1 (GH1), and has glucocerebrosidase activity.

### CROSS REFERENCE TO RELATED APPLICATIONS

This application is a U.S. National Phase Application under 35 U.S.C. 371 of International Application No. PCT/JP2021/004999, filed on Feb. 10, 2021, which claims priority to Japanese Patent Application No. 2020-088950, filed on May 21, 2020. The entire disclosures of the above applications are expressly incorporated by reference herein.

### SEQUENCE LISTING

This application contains references to amino acid sequences and/or nucleic acid sequences which have been submitted concurrently herewith as the sequence listing text file entitled “000014usnp_SequenceListing.TXT”, file size 19.7 KiloBytes (KB), created on Nov. 10, 2022. The aforementioned sequence listing is hereby incorporated by reference in its entirety pursuant to 37 C.F.R. § 1.52(e)(5).

### BACKGROUND

### Technical Field

The present invention relates to a protein having glucocerebrosidase activity and thermostability (hereinafter, this is also referred to as “thermostable glucocerebrosidase”), an enzyme composition, a pharmaceutical composition or a food composition including this protein, a method for producing ceramide using this protein, and the like.

### Related Art

Glucocerebrosidase is known as an enzyme that converts glucosylceramide, one of the glycolipids, to ceramide through hydrolysis. This glucocerebrosidase is found to exist mainly in animals and plays an important role in generating ceramide from glucosylceramide in the animal body, though its existence is hardly known for plants.

An inborn error of metabolism that cannot convert glucosylceramide in the body to ceramide due to congenital deficiency of glucocerebrosidase gene (Gaucher's disease) is known for humans. In this Gaucher's disease, glucosylceramide abnormally accumulates in the body so that various symptoms such as enlargement of the liver or the spleen, anemia, thrombocytopenia, and bone abnormality appear (Annual Review of Genomics and Human Genetics 4, 403-436 (2003) and British Journal of Haematology 129, 178-188 (2005)).

There reportedly exist Gaucher's disease patients equal to or more than approximately 5,000 people worldwide. Treatment that converts accumulated glucosylceramide to ceramide by replenishing scarce glucocerebrosidase with intravenous infusion drips in the body is typically performed as a treatment method therefor.

At present, imiglucerase which is human-derived glucocerebrosidase produced in Chinese hamster ovary cells by use of a gene recombination technique is typically used as an enzyme drug for use as intravenous infusion drips for Gaucher's disease. However, a problem of this imiglucerase is large economic burdens on patients because the drug price is high and furthermore, once-every-two weeks replenishment is required due to low thermostability.

Since animal-derived glucocerebrosidase is an enzyme that acts around the body temperature of the animal, any type thereof has low thermostability, as in the imiglucerase described above. Accordingly, if glucocerebrosidase that has thermostability and can minimize the number of times of replenishment is present, its medical value is unmeasurable. However, such glucocerebrosidase has not yet been reported.

Meanwhile, ceramide present in the stratum corneum of human skin, or the like is a component necessary for keeping moisture in the skin, and is blended into cosmetics or the like. This ceramide serving as an active ingredient in cosmetics or the like is usually produced by chemical synthesis. In this context, it could be possible to produce ceramide by decomposing relatively inexpensive glucosylceramide having a high content in animals or plants with glucocerebrosidase and to use this ceramide in cosmetics, reagents, or the like. However, since the imiglucerase described above is very expensive and also has low thermostability, practical use thereof has not yet been achieved. Unlike glucosylceramide, ceramide has a very low content in animals or plants. Therefore, direct extraction and purification of this ceramide involve enormous cost.

Accordingly, an object of the present invention is to provide a protein having glucocerebrosidase activity and further having thermostability.

### SUMMARY

The present inventor has conducted diligent studies to attain the object and found a protein which is derived from a plant, belongs to glycoside hydrolase family 1, and has glucocerebrosidase activity. It has been further found that this protein has thermostability, completing the present invention. It should be noted that there has been no report so far stating that glucocerebrosidase belonging to GH1 has been found from plants.

Specifically, the present invention relates to the following <1> to <17>.

<1> A protein which is derived from a plant, belongs to glycoside hydrolase family 1, and has glucocerebrosidase activity.
<2> The protein according to <1>, wherein the plant is a seed plant.
<3> The protein according to <2>, wherein the seed plant is one of a plant of the family Brassicaceae, a plant of the family Poaceae, a plant of the family Cucurbitaceae, a plant of the family Compositae, a plant of the family Solanaceae, a plant of the family Rosaceae, a plant of the family Amaryllidaceae, a plant of the family Leguminosae, and a plant of the family Liliaceae.
<4> A protein shown in the following (A), (B), or (C):
(A) a protein consisting of an amino acid sequence represented by SEQ ID NO: 1, amino acid positions 38 to 521 of SEQ ID NO: 1, SEQ ID NO: 2, or amino acid positions 19 to 503 of SEQ ID NO: 2 in the sequence listing.
(B) a protein which consists of the amino acid sequence represented by SEQ ID NO: 1, amino acid positions 38 to 521 of SEQ ID NO: 1, SEQ ID NO: 2, or amino acid positions 19 to 503 of SEQ ID NO: 2 in the sequence listing, having one or several nucleotides substituted, deleted, inserted, or added, belongs to glycoside hydrolase family 1, and has glucocerebrosidase activity and thermostability.
(C) a protein which has homology equal to or more than 60% to the amino acid sequence represented by SEQ ID NO: 1, amino acid positions 38 to 521 of SEQ ID NO: 1, SEQ ID NO: 2, or amino acid positions 19 to 503 of SEQ ID NO: 2 in the sequence listing, belongs to glycoside hydrolase family 1, and has glucocerebrosidase activity and thermostability.
<5> DNA encoding a protein according to any one of <1> to <4>.
<6> DNA shown in the following (a), (b), or (c):
(a) DNA which consists of a nucleotide sequence represented by SEQ ID NO: 3, nucleotide positions 112 to 1566 of SEQ ID NO: 3, SEQ ID NO: 4, or nucleotide positions 55 to 1512 of SEQ ID NO: 4 in the sequence listing, and encodes a protein belonging to glycoside hydrolase family 1 and having glucocerebrosidase activity.
(b) DNA which consists of the nucleotide sequence represented by SEQ ID NO: 3, nucleotide positions 112 to 1566 of SEQ ID NO: 3, SEQ ID NO: 4, or nucleotide positions 55 to 1512 of SEQ ID NO: 4 in the sequence listing, having one or several nucleotides substituted, deleted, inserted, or added, and encodes a protein belonging to glycoside hydrolase family 1 and having glucocerebrosidase activity and thermostability.
(c) DNA which is capable of hybridizing under stringent conditions to DNA consisting of a nucleotide sequence complementary to the nucleotide sequence represented by SEQ ID NO: 3, nucleotide positions 112 to 1566 of SEQ ID NO: 3, SEQ ID NO: 4, or nucleotide positions 55 to 1512 of SEQ ID NO: 4 in the sequence listing, and encodes a protein belonging to glycoside hydrolase family 1 and having glucocerebrosidase activity and thermostability.
<7> An expression vector for the expression of a protein belonging to glycoside hydrolase family 1 and having glucocerebrosidase activity and thermostability, the expression vector including DNA according to <5> or <6>.
<8> A transformant harboring an expression vector according to <7>.
<9> The transformant according to <8>, wherein the transformant is one of a plant, a plant cell, an animal cell, Escherichia coli, a yeast, and a filamentous fungus.
<10> A method for producing a protein, including the steps of: breeding or culturing a transformant according to <8> or <9>; and recovering a protein according to any one of <1> to <4> from the transformant or a material containing the transformant obtained by the step.
<11> An enzyme composition for glucosylceramide hydrolysis, containing a protein according to any one of <1> to <4>.
<12> A pharmaceutical composition including a protein according to any one of <1> to <4> as an active ingredient.
<13> The pharmaceutical composition according to <12> for Gaucher's disease prevention and treatment.
<14> A food composition containing a protein according to any one of <1> to <4>, and glucosylceramide isolated from a plant, an animal, or a microbe, or chemically synthesized glucosylceramide.
<15> A method for producing ceramide, including the step of using a protein according to any one of <1> to <4> to generate ceramide from glucosylceramide isolated from a plant, an animal, or a microbe, or chemically synthesized glucosylceramide.
<16> A method for preventing or treating Gaucher's disease, including administering (by intravenous infusion or the like) a protein according to any one of <1> to <4> or a pharmaceutical composition according to <12> or <13> to a Gaucher's disease patient.
<17> The method for preventing or treating Gaucher's disease according to <16>, wherein the protein or the pharmaceutical composition is administered to the Gaucher's disease patient once per 3 to 10 weeks such that glucocerebrosidase activity is 10 to 200 U/kg body weight.

### Effect of the Invention

The present invention can provide a protein belonging to glycoside hydrolase family 1 and having glucocerebrosidase activity and thermostability. Furthermore, an enzyme composition, a pharmaceutical composition, and a food composition comprising this protein can also be provided. Moreover, a method for producing ceramide using this protein can also be provided.

### BRIEF DESCRIPTION OF THE DRAWINGS

FIG. 1 is a graph showing the relationship between an incubation time at 37° C. (pH 5) and relative residual activity for rice-derived glucocerebrosidase (RGC1) and human-derived glucocerebrosidase (imiglucerase).

FIG. 2 is a graph showing the relationship between an incubation time at 37° C. (pH 7) and relative residual activity for rice-derived glucocerebrosidase (RGC1) and human-derived glucocerebrosidase (imiglucerase).

FIG. 3 is a graph showing the relationship between an incubation time at 45° C. (pH 5) and relative residual activity for soybean-derived glucocerebrosidase (SGC1) and human-derived glucocerebrosidase (imiglucerase).

FIG. 4 is a graph showing the relationship between an incubation time at 45° C. (pH 7) and relative residual activity for soybean-derived glucocerebrosidase (SGC1) and human-derived glucocerebrosidase (imiglucerase).

FIG. 5 shows the amino acid sequence of rice-derived glucocerebrosidase (RGC1).

FIG. 6 shows the nucleotide sequence of DNA encoding the rice-derived glucocerebrosidase (RGC1).

FIG. 7 shows the amino acid sequences of 9 peptide fragments obtained by the trypsin treatment of the rice-derived glucocerebrosidase (RGC1).

FIG. 8 shows the nucleotide sequences of a F-primer and a R-primer used in the cloning of the DNA encoding the rice-derived glucocerebrosidase (RGC1).

FIG. 9 shows the amino acid sequence of soybean-derived glucocerebrosidase (SGC1).

FIG. 10 shows the nucleotide sequence of DNA encoding the soybean-derived glucocerebrosidase (SGC1).

FIG. 11 shows the amino acid sequences of 7 peptide fragments obtained by the trypsin treatment of the soybean-derived glucocerebrosidase (SGC1).

FIG. 12 shows the nucleotide sequences of a F-primer and a R-primer used in the cloning of the DNA encoding the soybean-derived glucocerebrosidase (SGC1).

### DETAILED DESCRIPTION

The present invention will be described.

The present invention relates to a protein which is derived from a plant, belongs to glycoside hydrolase family 1, and has glucocerebrosidase activity (hereinafter, this is also referred to as the “protein of the present invention” or the “thermostable glucocerebrosidase of the present invention”), DNA encoding the protein of the present invention and an expression vector including this DNA, a transformant harboring this expression vector and a method for producing the protein of the present invention using this transformant, an enzyme composition, a pharmaceutical composition or a food composition including the protein of the present invention, a method for producing ceramide using the protein of the present invention, and the like.

First, the protein of the present invention will be described in detail.

The protein of the present invention is a protein which is derived from a plant, belongs to glycoside hydrolase family 1 (GH1), and has glucocerebrosidase activity. This protein is derived from a plant, belongs to GH1, and has glucocerebrosidase activity has thermostability.

In this context, the “glucocerebrosidase activity” is the enzymatic activity of an enzyme with EC No. of (EC3.2.1.45) (glucocerebrosidase), that is, the activity of catalyzing reaction of hydrolyzing the β-1,4-glycosyl bond between glucose and ceramide of the glycolipid glucosylceramide to generate ceramide. The “glycoside hydrolase family 1” is one of the carbohydrate hydrolase families classified into approximately 130 families according to Carbohydrate Active enzyme database (CAZy databe, http://www.cazy.org/). Glucocerebrosidase belonging to glycoside hydrolase family 1 (GH1), glucocerebrosidase belonging to glycoside hydrolase family 30 (GH30), and glucocerebrosidase belonging to glycoside hydrolase family 116 (GH116) are known.

The phrase “derived from a plant” means that the protein is expressed from a gene of the plant. This gene of the plant can be obtained from at least one selected from the group consisting of, for example, a leaf, a stem, a root, a seed, a fruit, a petal, a pistil, a pollen (stamen), a rhizoid, and a sporangium of the plant.

The “thermostability” means performance by which, when a solution containing a protein having glucocerebrosidase activity with glucocerebrosidase activity of 0.0002 to 0.0008 U/mL and a 50 mM acetate buffer solution (pH 5.0) that contain 0.1% Triton X-100 (registered trademark; the same holds true for the description below) and 0.05% sodium cholate is incubated at 37° C. for 30 hours, this glucocerebrosidase activity equal to or more than 80% remains (the relative residual activity equal to or more than 80% based on the glucocerebrosidase activity before incubation defined as 100%). For this thermostability, it is preferred that the relative residual activity described above be equal to or more than 90%. In this context, this glucocerebrosidase activity is measured as follows: a predetermined amount of sample is added to a 50 mM acetate buffer solution (pH 5.5) containing 100 μM glucosylceramide (cerebroside B, (4E,8E)-N-D-2′-hydroxypalmitoyl-1-O-β-D-glucopyranosyl-9-methyl-4,8-sphingadienine (The Journal of Antibiotics 41, (1988) 469-480; the same holds true for the description below), 0.1% Tween 20, and 0.05% sodium cholate, and incubated at 37° C. for 15 to 60 minutes. Subsequently, a 4-fold amount of ethanol is added to the obtained enzymatic reaction solution, mixed therewith, and then centrifuged at 15000 rpm for 20 minutes. The supernatant is subjected to high-performance liquid chromatography analysis to measure the amount of ceramide generated through enzymatic reaction. Conditions, other than those described above, necessary for the glucocerebrosidase activity measurement are carried out in accordance with a method described in Example 2 mentioned later.

The protein of the present invention is preferably derived from a seed plant. Specific examples of the seed plant include, but are not limited to, plants of the family Malvaceae, plants of the family Chenopodiaceae, plants of the family Rubiaceae, plants of the family Cannabaceae, plants of the family Hydrangeaceae, plants of the family Brassicaceae, plants of the family Iridaceae, plants of the family Poaceae, plants of the family Araliaceae, plants of the family Cucurbitaceae, plants of the family Anacardiaceae, plants of the family Cyperaceae, plants of the family Campanulaceae, plants of the family Compositae, plants of the family Lauraceae, plants of the family Moraceae, plants of the family Papaveraceae, plants of the family Araceae, plants of the family Cactaceae, plants of the family Lamiaceae, plants of the family Nymphaeaceae, plants of the family Apiaceae, plants of the family Polygonaceae, plants of the family Ericaceae, plants of the family Theaceae, plants of the family Solanaceae, plants of the family Caryophyllaceae, plants of the family Ulmaceae, plants of the family Nelumbonaceae, plants of the family Rosaceae, plants of the family Amaryllidaceae, plants of the family Convolvulaceae, plants of the family Vitaceae, plants of the family Fagaceae, plants of the family Paeoniaceae, plants of the family Leguminosae, plants of the family Rutaceae, plants of the family Pontederiaceae, plants of the family Oleaceae, plants of the family Arecaceae, plants of the family Salicaceae, plants of the family Liliaceae, plants of the family Orchidaceae, plants of the family Taxaceae, plants of the family Ginkgoaceae, plants of the family Taxodiaceae, plants of the family Cycadaceae, plants of the family Cupressaceae, and plants of the family Pinaceae.

Examples of the seed plant more specifically include cotton plant, hibiscus, spinach, fat hen, beet, madder, gardenia, coffee tree, marijuana, hop, hydrangea, Arabidopsis thaliana, rapeseed, Japanese radish, napa, cabbage, cauliflower, broccoli, Japanese mustard spinach, qing-geng-cai, wasabi, water iris, Japanese iris, iris, rice, timothy, wheat, maize, sorghum, barley, rye, sugar cane, oat, Japanese millet, foxtail millet, Korean lawn grass, common reed grass, bamboo, arrow bamboo, Japanese angelica tree, udo, fatsia, cucumber, bitter cucumber, melon, watermelon, bitter gourd, pumpkin, loofa, winter melon, gourd, calabash, lacquer tree, Japanese wax tree, star grass, balloon flower, goldenmane tickseed, lettuce, gerbera, gazania, thistle, burdock, sunflower, cosmos, dandelion, calendula, sweet coltsfoot, annual ragweed, camphor tree, bay tree, mulberry, fig, common poppy, water lettuce, eddo, cactus, Japanese basil, salvia, lavender, water lily, carrot, water celery, celery, buckwheat, polygonum, rhododendron, blueberry, alpine rose, camellia, tomato, eggplant, tobacco, petunia, capsicum, potato, dianthus, carnation, annual baby's breath, chickweed, Japanese zelkova, Muku tree, lotus, rose, cherry, almond, apricot, strawberry, Japanese apricot, apple, nashi pear, loquat, peach, garlic, green onion, onion, false bindweed, morning glory, sweet potato, grape, Japanese beech, jolcham oak, sawthorn oak, Japanese chestnut, tree peony, soybean, broad bean, black bean, Japanese wisteria, Japanese wisteria, lupine, kidney bean, pea, alfalfa, peanut, sweet pea, bird's-foot trefoil, miyagawa mandarin, Japanese pepper, Japanese summer orange, orange, lime, lemon, grapefruit, hardy orange, water hyacinth, olive, common jasmine, coconut palm, oil palm, Chinese date, chusan palm, poplar, basket willow, lily, morning star lily, tulip, narcissus, moth orchid, cattleya, vanilla, yew, gingko, cedar, cycad, Japanese cypress, and pine.

Among them, a protein that is derived from one of a plant of the family Brassicaceae, a plant of the family Poaceae, a plant of the family Cucurbitaceae, a plant of the family Compositae, a plant of the family Solanaceae, a plant of the family Rosaceae, a plant of the family Amaryllidaceae, a plant of the family Leguminosae, and a plant of the family Liliaceae, belongs to GH1, and has glucocerebrosidase activity is particularly suitable because of its excellent thermostability. For example, a rice-derived or soybean-derived protein that belongs to GH1 and has glucocerebrosidase activity has very suitable glucocerebrosidase activity and thermostability.

The rice-derived thermostable glucocerebrosidase of the present invention consists of the amino acid sequence represented by SEQ ID NO: 1 in the sequence listing, or the amino acid sequence represented by amino acid positions 38 to 521 of this SEQ ID NO: 1 which is an amino acid sequence from which a signal peptide has been cleaved off. The soybean-derived thermostable glucocerebrosidase of the present invention consists of the amino acid sequence represented by SEQ ID NO: 2 in the sequence listing, or the amino acid sequence represented by amino acid positions 19 to 503 of this SEQ ID NO: 2 which is an amino acid sequence from which a signal peptide has been cleaved off.

A protein which consists of the amino acid sequence represented by one of SEQ ID NO: 1 in the sequence listing, amino acid positions 38 to 521 of this SEQ ID NO: 1, SEQ ID NO: 2 in the sequence listing, and amino acid positions 19 to 503 of this SEQ ID NO: 2, having one or several nucleotides substituted, deleted, inserted, or added, belongs to GH1, and has glucocerebrosidase activity and thermostability is also encompassed in the present invention. In this case, this protein is preferably derived from a plant and may not be derived from a plant (the “protein of the present invention” mentioned later may include the one that is not derived from a plant). In this context, the term “several” means equal to or less than 10 and is preferably equal to or less than 6, more preferably equal to or less than 5.

This substitution, deletion, insertion, or addition of amino acids is conservative. That is, one or several amino acid residues are substituted, deleted, inserted, or added so as not to substantially alter the properties of the protein. Examples thereof include the case of substituting a hydrophobic amino acid residue with another hydrophobic amino acid residue, and the case of substituting a polar amino acid residue with another polar amino acid residue having the same charge thereas. Examples of such functionally similar amino acids specifically include hydrophobic (nonpolar) amino acids such as alanine, valine, isoleucine, leucine, proline, tryptophan, phenylalanine, and methionine. Among polar amino acids, examples of neutral amino acids include glycine, serine, threonine, tyrosine, glutamine, asparagine, and cysteine. Examples of basic amino acids include arginine, histidine, and lysine. Examples of acidic amino acids include aspartic acid and glutamic acid.

A protein which has homology equal to or more than 60%, more preferably equal to or more than 62%, further preferably equal to or more than 65%, further preferably equal to or more than 67%, further preferably equal to or more than 70%, further preferably equal to or more than 73%, further preferably equal to or more than 77%, to the amino acid sequence represented by one of SEQ ID NO: 1, amino acid positions 38 to 521 of SEQ ID NO: 1, SEQ ID NO: 2, and amino acid positions 19 to 503 of SEQ ID NO: 2 in the sequence listing, belongs to GH1, and has glucocerebrosidase activity and thermostability is also encompassed in the present invention. In this case as well, this protein is preferably derived from a plant and may not be derived from a plant. In this context, the “homology” is a numeric value calculated using default (initial setting) parameters in the homology search program EMBOSS Needle (https://www.ebi.ac.uk/Tools/psa/emboss needle/).

Next, DNA encoding the protein of the present invention, and a method for producing the protein using this DNA will be described in detail.

The DNA encoding the protein of the present invention may be naturally derived or synthesized using a portion of the naturally derived one, for example, without limitations as long as it is constituted by a nucleotide sequence that permits expression of this protein. For example, DNA consisting of a nucleotide sequence represented by SEQ ID NO: 3 or nucleotide positions 112 to 1566 of SEQ ID NO: 3 in the sequence listing is disclosed as DNA encoding the rice-derived thermostable glucocerebrosidase of the present invention mentioned above. DNA consisting of a nucleotide sequence represented by SEQ ID NO: 4 or nucleotide positions 55 to 1512 of SEQ ID NO: 4 in the sequence listing is disclosed as DNA encoding the soybean-derived thermostable glucocerebrosidase of the present invention mentioned above.

Further examples thereof also include DNA which consists of the nucleotide sequence represented by one of SEQ ID NO: 3, nucleotide positions 112 to 1566 of SEQ ID NO: 3, SEQ ID NO: 4, and nucleotide positions 55 to 1512 of SEQ ID NO: 4 in the sequence listing, having one or several nucleotides substituted, deleted, inserted, or added, and encodes a protein belonging to GH1 and having glucocerebrosidase activity and thermostability. In this context, the term “several” means equal to or less than 20 and is preferably equal to or less than 10, more preferably equal to or less than 6.

Further examples thereof also include DNA which is capable of hybridizing under stringent conditions to DNA consisting of a nucleotide sequence complementary to the nucleotide sequence represented by one of SEQ ID NO: 3, nucleotide positions 112 to 1566 of SEQ ID NO: 3, SEQ ID NO: 4, and nucleotide positions 55 to 1512 of SEQ ID NO: 4 in the sequence listing, and encodes a protein belonging to GH1 and having glucocerebrosidase activity and thermostability.

In this context, the “stringent conditions” are conditions under which a so-called specific hybrid is formed and a nonspecific hybrid is not formed. One example thereof includes conditions involving performing hybridization at 65° C. in the presence of 0.7 to 1.0 M sodium chloride, and then performing washing one to three times at 60° C., preferably 65° C., more preferably 68° C., using a solution of 0.1 to 5×SSC and 0.1% SDS (composition of 1×SSC: 150 mM sodium chloride and 15 mM sodium citrate).

The present invention also provides an expression vector that is capable of replicating in a plant, a plant cell, an animal cell (also including an insect cell), or a microbe serving as a host and includes the DNA mentioned above in a state that permits expression of the protein encoded thereby. That is, an expression vector for the expression of a protein that belongs to GH1 and has glucocerebrosidase activity and thermostability is also provided. This expression vector can be constructed using, for example, a self-replicating vector which resides extrachromosomally in an independent form in a cell of a host and replicates in a manner independent of the replication of chromosomal DNA, or a vector that is integrated into the chromosomal DNA of a cell of a host and replicates together with this chromosomal DNA. Preferred examples thereof include plasmid vectors and virus vectors. A method that is routinely used in the field of gene engineering can be used as procedures and a method for constructing the expression vector.

This expression vector preferably contains, in addition to the DNA encoding the protein of the present invention, a nucleotide sequence that controls expression thereof, a gene marker for the selection of a transformant, and the like in order to express the protein of the present invention in a transformant actually harboring this. Examples of the nucleotide sequence that controls expression include promoters, terminators, and nucleotide sequences encoding signal peptides other than those mentioned above. The promoter is not particularly limited as long as it exhibits transcriptional activity in a host. The signal peptide is not particularly limited as long as it contributes to the secretion of the protein to the outside of a cell in a host, for example. The gene marker can be appropriately selected according to a method for selecting a transformant. For example, a drug resistance gene or a gene that compensates for auxotrophy can be used.

The present invention also provides a transformant harboring the expression vector described above in a cell and/or chromosomal DNA of a host. This host-vector system is not particularly limited. For example, a system using a plant, a plant cell, an animal cell, or a microbe (Escherichia coli, yeast, filamentous fungus, or the like), or a fusion protein expression system with another protein using one of them can be used. The transfer of the expression vector described above to a host, that is, the transformation of a host using the expression vector described above, can be carried out in accordance with a method that is routinely used in the art. In this context, any of a plant, a plant cell, an animal cell, Escherichia coli, a yeast, and a filamentous fungus is preferably used as the host to be transformed. That is, a transformant of any of a plant, a plant cell, an animal cell, Escherichia coli, a yeast, and a filamentous fungus harboring the expression vector described above is preferred. For example, the transformant of a plant, a plant cell, or Escherichia coli harboring the expression vector described above can be allowed to express the protein of the present invention in a large amount.

This transformant is bred or cultured under conditions that permit growth and proliferation with its trait maintained, and the protein of the present invention can be recovered from the obtained transformant (plant, cultured plant cell, cultured animal cell, or the like) or material containing it (culture solution, solid medium, or the like containing the cultured cell). Thus, the present invention provides a method for producing the protein of the present invention, including the steps of: breeding or culturing the transformant; and recovering (crudely processing or purifying) the protein of the present invention from a transformant or a material containing it obtained by the step. A method for breeding or culturing the transformant and conditions therefor can be a method and conditions that permit growth and proliferation of the transformant with its trait maintained, without particular limitations, and may be substantially equivalent to a breeding or culture method and conditions as to the plant, the plant cell, the animal cell, or the microbe for use as a host. A crude processing method or a purification method that is routinely used in the art can also be used as a method for recovering the protein of interest after breeding or culture of the transformant.

One example of a preferred embodiment of the method for producing the protein of the present invention includes a method using a transformant of a plant, a plant cell, or an animal cell. Examples of the animal cell include Chinese hamster and human cells. Examples of the plant or the plant cell include plants of the family Malvaceae, plants of the family Chenopodiaceae, plants of the family Rubiaceae, plants of the family Cannabaceae, plants of the family Hydrangeaceae, plants of the family Brassicaceae, plants of the family Iridaceae, plants of the family Poaceae, plants of the family Araliaceae, plants of the family Cucurbitaceae, plants of the family Anacardiaceae, plants of the family Cyperaceae, plants of the family Campanulaceae, plants of the family Compositae, plants of the family Lauraceae, plants of the family Moraceae, plants of the family Papaveraceae, plants of the family Araceae, plants of the family Cactaceae, plants of the family Lamiaceae, plants of the family Nymphaeaceae, plants of the family Apiaceae, plants of the family Polygonaceae, plants of the family Ericaceae, plants of the family Theaceae, plants of the family Solanaceae, plants of the family Caryophyllaceae, plants of the family Ulmaceae, plants of the family Nelumbonaceae, plants of the family Rosaceae, plants of the family Amaryllidaceae, plants of the family Convolvulaceae, plants of the family Vitaceae, plants of the family Fagaceae, plants of the family Paeoniaceae, plants of the family Leguminosae, plants of the family Rutaceae, plants of the family Pontederiaceae, plants of the family Oleaceae, plants of the family Arecaceae, plants of the family Salicaceae, plants of the family Liliaceae, plants of the family Orchidaceae, plants of the family Taxaceae, plants of the family Ginkgoaceae, plants of the family Taxodiaceae, plants of the family Cycadaceae, plants of the family Cupressaceae, and plants of the family Pinaceae, and their cells. Examples thereof more specifically include cotton plant, hibiscus, spinach, fat hen, beet, madder, gardenia, coffee tree, marijuana, hop, hydrangea, Arabidopsis thaliana, rapeseed, Japanese radish, napa, cabbage, cauliflower, broccoli, Japanese mustard spinach, qing-geng-cai, wasabi, water iris, Japanese iris, iris, rice, timothy, wheat, maize, sorghum, barley, rye, sugar cane, oat, Japanese millet, foxtail millet, Korean lawn grass, common reed grass, bamboo, arrow bamboo, Japanese angelica tree, udo, fatsia, cucumber, bitter cucumber, melon, watermelon, bitter gourd, pumpkin, loofa, winter melon, gourd, calabash, lacquer tree, Japanese wax tree, star grass, balloon flower, goldenmane tickseed, lettuce, gerbera, gazania, thistle, burdock, sunflower, cosmos, dandelion, calendula, sweet coltsfoot, annual ragweed, camphor tree, bay tree, mulberry, fig, common poppy, water lettuce, eddo, cactus, Japanese basil, salvia, lavender, water lily, carrot, water celery, celery, buckwheat, polygonum, rhododendron, blueberry, alpine rose, camellia, tomato, eggplant, tobacco, petunia, capsicum, potato, dianthus, carnation, annual baby's breath, chickweed, Japanese zelkova, Muku tree, lotus, rose, cherry, almond, apricot, strawberry, Japanese apricot, apple, nashi pear, loquat, peach, garlic, green onion, onion, false bindweed, morning glory, sweet potato, grape, Japanese beech, jolcham oak, sawthorn oak, Japanese chestnut, tree peony, soybean, broad bean, black bean, Japanese wisteria, Japanese wisteria, lupine, kidney bean, pea, alfalfa, peanut, sweet pea, bird's-foot trefoil, miyagawa mandarin, Japanese pepper, Japanese summer orange, orange, lime, lemon, grapefruit, hardy orange, water hyacinth, olive, common jasmine, coconut palm, oil palm, Chinese date, chusan palm, poplar, basket willow, lily, morning star lily, tulip, narcissus, moth orchid, cattleya, vanilla, yew, gingko, cedar, cycad, Japanese cypress, pine, and their cells.

Another example of a preferred embodiment of the method for producing the protein of the present invention includes a method using a transformant of an Escherichia coli cell, a yeast cell, or a filamentous fungus cell. Examples of the yeast cell include cells of microbes belonging to the genus Saccharomyces, the genus Hansenula, or the genus Pichia, for example, Saccharomyces cerevisiae. Examples of the filamentous fungus cell include cells of ones belonging to the genus Humicola, the genus Trichoderma, the genus Staphylotrichum, the genus Aspergillus, the genus Fusarium, or the genus Acremonium.

Next, an enzyme composition, a pharmaceutical composition, and a food composition including the protein of the present invention, and the like will be described in detail.

The present invention can provide an enzyme composition containing the protein of the present invention mentioned above. This enzyme composition includes the protein of the present invention as an active ingredient (active component of the glucocerebrosidase activity of the enzyme composition) and can be suitably used for glucosylceramide hydrolysis (for the hydrolysis of glucose in a glucosylceramide molecule), that is, for the conversion of glucosylceramide to ceramide.

The protein of the present invention can be included as an active ingredient to thereby provide a pharmaceutical composition. This pharmaceutical composition can be suitably used for the prevention and treatment of Gaucher's disease. Its form is preferably intravenous infusion drips and may be an oral drug (tablet, powder, syrup, or the like).

In other words, the present invention can provide a method for preventing or treating Gaucher's disease, including administering (for example, by intravenous infusion) a pharmaceutical composition including the protein of the present invention as an active ingredient to a Gaucher's disease patient. Its dosage and administration suitably involve administering the protein of the present invention or the pharmaceutical composition (prophylactic and therapeutic agent for Gaucher's disease) including the protein of the present invention as an active ingredient to a Gaucher's disease patient once per 3 to 10 weeks, more preferably once per 4 to 8 weeks, such that glucocerebrosidase activity is 10 to 200 U/kg body weight. In this case, the term “U (unit)” is a unit at which 1 μmol of a synthetic substrate p-nitrophenyl-β-D-glucopyranoside is decomposed at 37° C. for 1 minute.

The protein of the present invention and glucosylceramide isolated from a plant, an animal, or a microbe (for example, basidiomycete) or chemically synthesized glucosylceramide can be included to thereby provide a food composition that can easily liberate ceramide from the glucosylceramide in the composition. Furthermore, the protein of the present invention and the glucosylceramide can also be included as an active ingredient to thereby provide a food composition for at least one purpose selected from improvement in skin moisture, prevention of skin damage caused by ultraviolet ray, prevention of inflammatory bowel disease, and prevention of colorectal cancer as a functional food in which this ceramide liberated serves as a functional material. Moreover, a food composition for the prevention of lifestyle-related disease (e.g., diabetes mellitus, heart disease, hypertension, and hyperlipemia) can also be provided.

According to the present invention, the thermostable glucocerebrosidase of the present invention can also be included as an active ingredient to thereby provide a food composition for Gaucher's disease prevention or treatment as a functional food, or a food composition for ceramide supplementation which is ingested with a food composition in which glucosylceramide is included. The dosage and administration of the food composition for Gaucher's disease described above can be the same as those of the pharmaceutical composition for the prevention and treatment of Gaucher's disease mentioned above.

Next, a method for producing ceramide using the protein of the present invention will be described in detail.

The method for producing ceramide using the protein of the present invention includes the step of using the protein of the present invention to generate ceramide from glucosylceramide isolated from a plant, an animal, or a microbe (for example, basidiomycete) or chemically synthesized glucosylceramide. This production method may include an arbitrary step other than those described above without largely influencing the effect of the present invention.

Glucosylceramide is relatively abundant in organisms such as animals or plants, as compared with ceramide. Ceramide can be produced at low cost from such glucosylceramide by using the thermostable glucocerebrosidase of the present invention which is inexpensively obtained by expression in a large amount by the transformant mentioned above. Furthermore, the ceramide thus obtained can be used to inexpensively provide a cosmetic or a functional food aimed at an improving effect on the retention of skin moisture, a preventive effect on skin damage caused by ultraviolet ray, a preventive effect on colorectal cancer, or the like. Moreover, ceramide as a reagent for research or a drug can also be inexpensively provided.

The embodiments described above are mere examples for facilitating understanding of the present invention and do not limit the present invention.

Hereinafter, Examples of the present invention will be described. However, the present invention is not limited by Examples given below, and various changes or modifications can be made within the technical brief of the present invention.

### EXAMPLES

### Example 1

10 g of young leaves of rice (Oryza sativa L. cv. Nipponbare) was collected and homogenized with a homogenizer in a 50 mM acetate buffer solution (pH 5.5) containing 0.05% sodium cholate and 0.3% Triton X-100. This homogenate was centrifuged for 20 minutes under conditions of 15000 rpm and 4° C. The centrifuged supernatant was dialyzed using a 1000-fold amount of a 40 mM sodium acetate buffer solution (pH 5.5) containing 0.05% Tween 20 (registered trademark; the same holds true for the description below) and 0.025% sodium cholate to obtain an enzyme extract. To 50 mL of this enzyme extract, 10 mM glucosylceramide (cerebroside C, (4E,8E)-N-D-2′-hydroxy-(E)-3′-octadecenoyl-1-O-β-D-glucopyranosyl-9-methyl-4,8-sphingadienine (The Journal of Antibiotics 41, (1988)469-480; the same holds true for the description below) dissolved in 1 mL of ethanol, and 49 mL of 0.4% sodium cholate were added, followed by reaction at 45° C. for 5 hours. The obtained reaction solution was adjusted to pH 11.5 with a sodium carbonate solution and a sodium hydroxide solution. Then, an ethyl acetate solution was added and mixed therewith, and centrifuged for 20 minutes under conditions of 3000 rpm and 4° C. After the centrifugation, the obtained ethyl acetate layer was dried. Then, this dried product was dissolved in an 80% ethanol solution and subjected to high-performance liquid chromatography analysis. Specifically, the sample was injected to TSKgel ODS-120T column (4.6 mm×30 cm, manufactured by Tosoh Corp., registered trademark (the same holds true for the description below)), and a solvent having an ethanol concentration of 81% was flowed at a flow rate of 1.0 mL/min, followed by detection in a UV detector (ultraviolet absorption wavelength: 215 nm) to thereby separate a substance newly generated through enzymatic reaction.

Then, IR (FTS-135, manufactured by Bio-Rad Laboratories, Inc.), ¹H NMR, ¹³C NMR (Varian UNITY plus 500 spectrometer, manufactured by Agilent Technologies, Inc.), and ESI-MS analysis (Agilent 6460, manufactured by Agilent Technologies, Inc.) of the separated substance were conducted. The results are shown in Table 1 below.

TABLE 1
IR
Vₘₐₓ 2921, 2851, 1733, 1650, 1538, 1468, 1379, 1288, 1124, 1076, 1033, 961 cm⁻¹
¹H NMR (CD₃OD)
δ 0.90 (6H, t, J = 6.87 Hz, Me-18,18′), 1.29 (34H, m, H-12-17, H-7′-17′), 1.37-1.42 (4H, m, H-11, 6′), 1.60
(3H, s, Me-19), 1.98 (2H, m, H-10), 2.02 (2H, m, H-5′), 2.02 (2H, m, H-6), 2.08 (2H, m, H-7), 3.67 (1H, dd,
J = 10.83, 3.66 Hz, H-1), 3.79 (1H, dd, J = 10.83, 5.19 Hz, H-1), 3.83 (1H, m, J = 7.02, 5.19 Hz, H-2), 4.07 (1H,
t, J = 7.17, 7.02 Hz, H-3), 4.43 (1H, d, J = 6.26 Hz, H-2′), 5.14 (1H, t, J = 6.71 Hz, H-8), 5.47 (1H, dd, J = 15.41,
7.17 Hz, H-4), 5.50 (1H, dd, J = 15.26, 6.26 Hz, H-3′), 5.71 (1H, dt, J = 15.26 Hz, H-4′), 5.83 (1H, dt,
J = 15.41, 6.87 Hz, H-5)
¹³C NMR (125 MHz, CD₃OD)
δ 14.5 [C-18, −18′], 16.2 [C-19], 23.8 [C-17, −17′], 28.8 [C-7], 29.2 [C-11], 30.3-30.9 [C-12~15,C-6′~15′],
33.1 [C-16, −16′], 33.5 [C-5′], 33.8 [C-6], 40.8 [C-10], 56.1 [C-2], 61.9 [C-1], 73.4 [C-3], 74.1 [C-2′], 124.9
[C-8], 129.2 [C-3′], 131.2 [C-4], 134.4 [C-5], 134.9 [C-4′], 136.8 [C-9], 175.5 [C-1′1] Parts per million
from TMS using center peak of CD₃OD at 49.0 ppm.
Negative ESI-MS m/z: 590.5 [M − H]⁻

The results revealed that the substance generated from cerebroside C by the rice-derived enzyme described above was ceramide derived from the cerebroside C. This substance was completely consistent in terms of the IR, ¹H NMR, ¹³C NMR, and ESI-MS analysis results with ceramide generated from cerebroside C by imiglucerase (manufactured by Sanofi S. A.), human-derived glucocerebrosidase. These results revealed that this rice-derived enzyme had glucocerebrosidase activity.

### Example 2

110 g of stems of rice (Oryza sativa L. cv. Nipponbare) was collected, chopped, and then homogenized with a homogenizer in a 20 mM acetate buffer solution (pH 5.5) containing 0.05% sodium cholate. This homogenate was centrifuged for 60 minutes under conditions of 10000 rpm and 4° C. The centrifuged supernatant was removed. Further, a 20 mM acetate buffer solution (pH 5.5) containing 0.05% sodium cholate was added to the centrifuged precipitate, stirred for 5 minutes, and then centrifuged for 60 minutes under conditions of 10000 rpm and 4° C. The centrifuged supernatant was removed. Further, a 20 mM acetate buffer solution (pH 5.5) containing 0.05% sodium cholate was added to the centrifuged precipitate, and Triton X-100 was added thereto in an amount sufficient for the elution of glucocerebrosidase. After stirring for 30 minutes, centrifugation was performed for 60 minutes under conditions of 18000 rpm and 4° C. The obtained centrifuged supernatant was filtered through Durapore Membrane Filter 0.45 μm HV (manufactured by Merck Millipore, registered trademark (the same holds true for the description below)), and then desalted and concentrated to obtain an enzyme extract.

This enzyme extract was applied to HiTrap Q HP column (manufactured by Amersham Biosciences Corp., registered trademark (the same holds true for the description below)) equilibrated with a 20 mM acetate buffer solution (pH 6.0) containing 0.05% sodium cholate and 0.1% Triton X-100. Then, elution was performed by the gradient elution method from the 20 mM acetate buffer solution (pH 6.0) containing 0.05% sodium cholate and 0.1% Triton X-100 into a buffer solution containing 1 M sodium chloride in a 20 mM acetate buffer solution (pH 6.0) containing 0.05% sodium cholate and 0.1% Triton X-100. Next, a fraction with strongly observed glucocerebrosidase activity was pooled, and desalted and concentrated by ultrafiltration. This desalted and concentrated solution was applied to HiTrap SP HP column (manufactured by Amersham Biosciences Corp.) equilibrated with a 20 mM acetate buffer solution (pH 5.5) containing 0.05% sodium cholate and 0.1% Triton X-100. Then, elution was performed for fractionation by the gradient elution method from the 20 mM acetate buffer solution (pH 5.5) containing 0.05% sodium cholate and 0.1% Triton X-100 into a buffer solution containing 1 M sodium chloride in a 20 mM acetate buffer solution (pH 5.78) containing 0.05% sodium cholate and 0.1% Triton X-100. Then, an enzyme fraction with strongly observed glucocerebrosidase activity was pooled, and desalted and concentrated by ultrafiltration. The resulting solution was used as rice-derived glucocerebrosidase (RGC1).

The glucocerebrosidase activity of the fraction was measured as follows: a predetermined amount of sample was added to a 50 mM acetate buffer solution (pH 5.5) containing 100 μM glucosylceramide (cerebroside B), 0.1% Tween 20, and 0.05% sodium cholate, and incubated at 37° C. for 15 minutes. Subsequently, a 4-fold amount of ethanol was added to the obtained reaction solution, mixed therewith, and then centrifuged at 15000 rpm for 20 minutes. The centrifuged supernatant was subjected to high-performance liquid chromatography analysis. In the high-performance liquid chromatography analysis, the sample was injected to TSKgel ODS-120T column (4.6 mm×30 cm), and a solvent having an ethanol concentration of 83% was flowed at a flow rate of 0.8 mL/min, followed by detection in a UV detector (ultraviolet absorption wavelength: 215 nm) to thereby measure the amount of ceramide newly generated through enzymatic reaction. The amount of ceramide was determined by using, as a standard, ceramide generated from cerebroside B by imiglucerase (manufactured by Sanofi S. A.), human-derived glucocerebrosidase. The molecular weight of the generated ceramide was examined by negative LC-MS (manufactured by Agilent Technologies, Inc.) to thereby confirm that it was ceramide generated from cerebroside B (ESI-MS m/z: 564.5[M-H]⁻). Glucocerebrosidase activity per mL of the enzyme solution was calculated when the amount of the enzyme that generated 1 μmol of ceramide for 1 minute in the enzymatic reaction solution was defined as 1 unit (U).

The protein concentration of the purified RGC1 was determined in Protein Assay Kit (manufactured by Bio-Rad Laboratories, Inc.) by using bovine serum albumin as a standard and imiglucerase whose protein concentration was measured in advance as a preparation. Specifically, the purified RGC1 solution and the imiglucerase solution were each injected to TSKgel Octyl-80 Ts column (4.6 mm×15 cm, manufactured by Tosoh Corp.), and solutions of acetonitrile in 0.05% trifluoroacetic acid were flowed in a gradient mode of elevated acetonitrile ratios at a flow rate of 0.8 mL/min, followed by detection in a UV detector (ultraviolet absorption wavelength: 280 nm). The resulting peak area was determined and compared with imiglucerase to thereby determine the protein concentration of the purified RGC1.

The fraction containing this RGC1 exhibited a single band in SDS-PAGE, and its average molecular weight (MW) was approximately 62 kD. The SDS-PAGE was performed using AE-6000 electrophoresis (manufactured by ATTO Corp.) and Precast Minigel e-PAGEL (E-R10L/gel concentration of 10%/18 samples, manufactured by ATTO Corp.). Silver staining was performed with Silver Stain MS Kit (manufactured by FUJIFILM Wako Pure Chemical Corp.). The molecular weight marker used was SDS-PAGE Molecular Weight Standards Low Range (manufactured by Bio-Rad Laboratories, Inc.).

### Example 3

RGC1 obtained in Example 2 was added to 12.5 μM glucosylceramide (one of the 12 animal-derived, plant-derived, or filamentous fungus-derived ones in Table 2 below) or synthetic β-glucoside, and a 50 mM acetate buffer solution (pH 5.5) containing 0.1% Tween 20 and 0.05% sodium cholate, and incubated at 37° C. for 15 minutes. Subsequently, a 4-fold amount of ethanol was added to the obtained reaction solution, mixed therewith, and then centrifuged under a condition of 15000 rpm for 20 minutes. The amount of ceramide in the supernatant was measured by high-performance liquid chromatography using various reverse-phase columns to thereby determine glucocerebrosidase activity. The amount of ceramide was determined by using, as a standard, ceramide generated from the glucosylceramide by imiglucerase. The glucocerebrosidase activity was determined as relative activity when activity in the case of using animal-derived glucosylceramide d18:1(4E)-C8:0-GluCer as a substrate was defined as 100 (rightmost column of Table 2 below). This test was conducted five times, and average relative activity was determined therefrom. The results are shown in Table 2 below.

TABLE 2

| | | Fatty | Sphingosine | Relative |
|---|---|---|---|---|
| Type of substrate | Sugar | acid | base | activity (%) |
| Animal-derived | | | | |
| d18:1(4E)-C18:0-GM₃ | NeuAc-Gal-Glu | 18:0 | d18:1(4E) | ND |
| d18:1(4E)-C8:0-LacCer | Lac | 8:0 | d18:1(4E) | 3.4 |
| d18:1(4E)-C8:0-GalCer | Gal | 8:0 | d18:1(4E) | 5.8 |
| d18:1(4E)-C8:0-GluCer | Glu | 8:0 | d18:1(4E) | 100 |
| d18:1(4E)-C12:0-GluCer | Glu | 12:0 | d18:1(4E) | 82.9 |
| d18:1(4E)-C18:0-GluCer | Glu | 18:0 | d18:1(4E) | 50.4 |
| Plant-derived | | | | |
| d18:2(4E,8Z)-C16h:0-GluCer | Glu | 16h:0 | d18:2(4E,8Z) | 147.7 |
| d18:2(4E,8E)-C16h:0-GluCer | Glu | 16h:0 | d18:2(4E,8E) | 96.1 |
| d18:2(4E,8Z)-C18h:0-GluCer | Glu | 18h:0 | d18:2(4E,8Z) | 143.3 |
| Filamentous fungus-derived | | | | |
| d19:2(4E,8E,9Me)-C16h:0-GluCer | Glu | 16h:0 | d19:2(4E,8E,9Me) | 116.7 |
| (cerebroside B) | | | | |
| d19:2(4E,8E,9Me)-C16h:1-GluCer | Glu | 16h:1 | d19:2(4E,8E,9Me) | 117.7 |
| d19:2(4E,8E,9Me)-C18h:1-GluCer | Glu | 18h:1 | d19:2(4E,8E,9Me) | 113.2 |
| (cerebroside C) | | | | |
| Synthetic β-glucoside | | | | |
| pNP-β-glucoside | Glu | — | — | 2.5 |

Glu, glucose;
Lac, lactose;
Gal, galactose;
NeuAc, acetylneuraminic acid;
Cer, ceramide;
GluCer, glucosylceramide;
GalCer, galactosylceramide;
LacGer, lactosylceramide;
16:0, palmitic acid;
16:1, (E)-3-hexadecenoic acid;
16h:1, 2-hydroxy-(E)-3-hexadecenoic acid;
18:0, stearic acid;
18:1, (E)-3-octadecenoic acid;
18h:1, 2-hydroxy-(E)-3-octadecenoic acid;
d18:1(4E), (E)-4-sphingenine;
d18:2(4E,8E), (4E,8E)-4,8-sphingadienine;
d19:2(4E,8E,9Me), (4E,8E)-9-methyl-4,8-sphingadienine;
pNP-β-glucoside, 4-nitrophenyl-β-D-glucopyranoside

As a result, RGC1 rarely reacted when lactosylceramide such as d18:1(4E)-C18:0-GM₃ or d18:1(4E)-C8:0-LacCer and galactosylceramide such as d18:1(4E)-C8:0-GalCer were used as substrates, revealing that it reacted by specifically recognizing the glucose structure of glucosylceramide. Further, RGC1 also rarely reacted with pNP-β-glucoside, revealing that it reacted by specifically recognizing the ceramide structure of glucosylceramide. From these results, RGC1 was found to be glucocerebrosidase. It was also shown that RGC1 also reacted with any type of glucosylceramide substrate derived from a plant, an animal, or a filamentous fungus.

### Example 4

Four types, glucocerebrosidase 1 (GBA1; imiglucerase, belonging to GH30), glucocerebrosidase 2 (GBA2, belonging to GH116), glucocerebrosidase 3 (GBA3, belonging to GH1), and lactase-phlorizin hydrolase (belonging to GH1), are known as animal-derived glucocerebrosidase. Glucocerebrosidase belonging to GH116 (AtGCD3) has been isolated as plant-derived glucocerebrosidase from thale-cress. Accordingly, the K_{cat}/Kₘ values (values obtained by dividing the number of molecules of a substrate that can be converted for 1 second by one molecule of an enzyme by a substrate concentration that provides a reaction rate of 50% of the maximum reaction rate of the enzyme) of RGC1 obtained in Example 2 and imiglucerase were determined by the Hanes-Woolf plot in accordance with the reaction method of Example 2 using a synthetic glucosylceramide substrate N-[6-[(7-nitro-2-1,3-benzoxadiazol-4-yl)amino]hexanoyl]-D-glucosyl-β1-1′-sphingosine (C6-NBD glucosylceramide). The K_{cat}/Kₘ values of animal-derived glucocerebrosidase GBA3 and thale-cress-derived glucocerebrosidase AtGCD3 were taken from documented values (Journal of Biological Chemistry 282, (2007) 30889-30900, and Journal of Biological Chemistry 295, (2020) 717-728). The results are shown in Table 3 below. From the results, RGC1 had an evidently high K_{cat}/Kₘ value compared with other glucocerebrosidases, revealing that it was excellent glucocerebrosidase.

TABLE 3

| | K_{cat}/Kₘ |
|---|---|
| | (s⁻¹/μM) |
| RGC1 | 1.97 |
| Imiglucerase | 0.419 |
| (GBA1) | |
| GBA3 | 0.0262 |
| AtGCD3 | 0.0053 |

### Example 5

RGC1 obtained in Example 2 and imiglucerase were each added to a 50 mM acetate buffer solution (pH 5.5) containing 100 μM glucosylceramide (cerebroside B), 0.1% Tween 20, and 0.05% sodium cholate, and incubated at each temperature for 15 minutes. Subsequently, a 4-fold amount of ethanol was added to the obtained reaction solution, mixed therewith, and then centrifuged under a condition of 15000 rpm for 20 minutes. The supernatant was subjected to high-performance liquid chromatography analysis to measure the amount of ceramide generated through enzymatic reaction. The high-performance liquid chromatography analysis was carried out in the same manner as in Example 2. The amount of ceramide and enzymatic activity were also calculated in the same manner as in Example 2. A temperature that attained the highest glucocerebrosidase activity was regarded as the optimum temperature. This test was conducted three times, and an average value was determined therefrom. The results are shown in Table 4 below.

From the results, the optimum temperature of the human-derived glucocerebrosidase imiglucerase was 42.5° C., whereas the optimum temperature of the rice-derived glucocerebrosidase RGC1 was 54.0° C., suggesting that RGC1 was highly stable in vivo.

TABLE 4

| | Optimum |
|---|---|
| | temperature (° C.) |
| Imiglucerase | 42.5 |
| RGC1 | 54.0 |

### Example 6

For enzymes, it is generally known that a higher optimum temperature leads to better thermostability. From Example 5, RGC1 was expected to have thermostability because its optimum temperature for glucocerebrosidase activity was higher than that of imiglucerase. Accordingly, the following test was carried out in order to confirm thermostability in lysosome (pH 5) and cytoplasm (pH 7) where glucocerebrosidase typically resides and acts in vivo in humans.

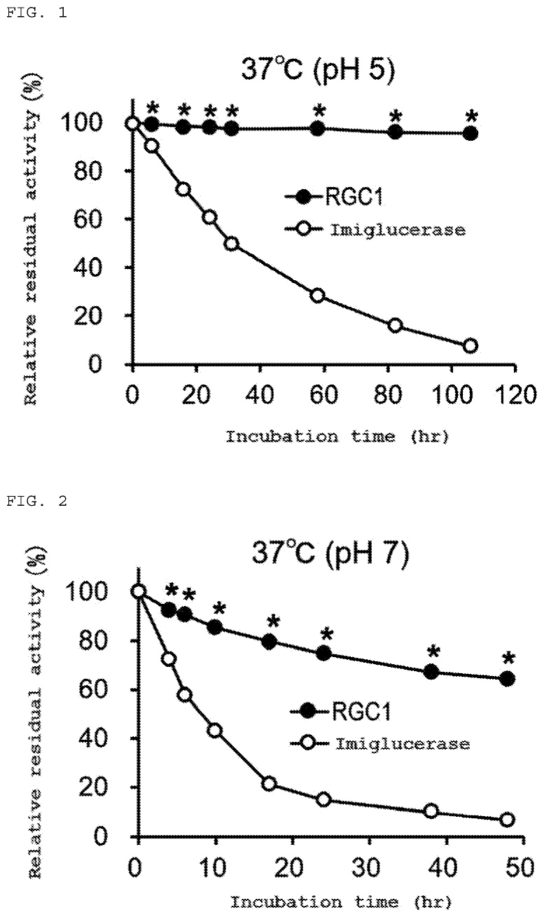

RGC1 obtained in Example 2 and imiglucerase were each incubated at pH 5 simulating lysosome (50 mM acetate buffer solution (pH 5.0) containing 0.1% Triton X-100 and 0.05% sodium cholate) at 37° C. for 6 to 106 hours or at pH 7 simulating cytoplasm (50 mM potassium phosphate buffer solution (pH 7.0) containing 0.1% Triton X-100 and 0.05% sodium cholate) at 37° C. for 4 to 48 hours. Glucocerebrosidase activity after the incubation for each time was measured in accordance with the method of Example 2. Residual activity was calculated as a relative activity value when activity before incubation was defined as 100. This test was conducted three times, and an average value was determined therefrom. The results are shown in FIGS. 1 and 2. In these figures, the mark * was added when the glucocerebrosidase activity of RGC1 thus reacted for each time was significantly higher than that of imiglucerase (1% significance).

From FIGS. 1 and 2, RGC1 was evidently superior in stability to imiglucerase at both pH 5 and pH 7, indicating that it had thermostability.

### Example 7

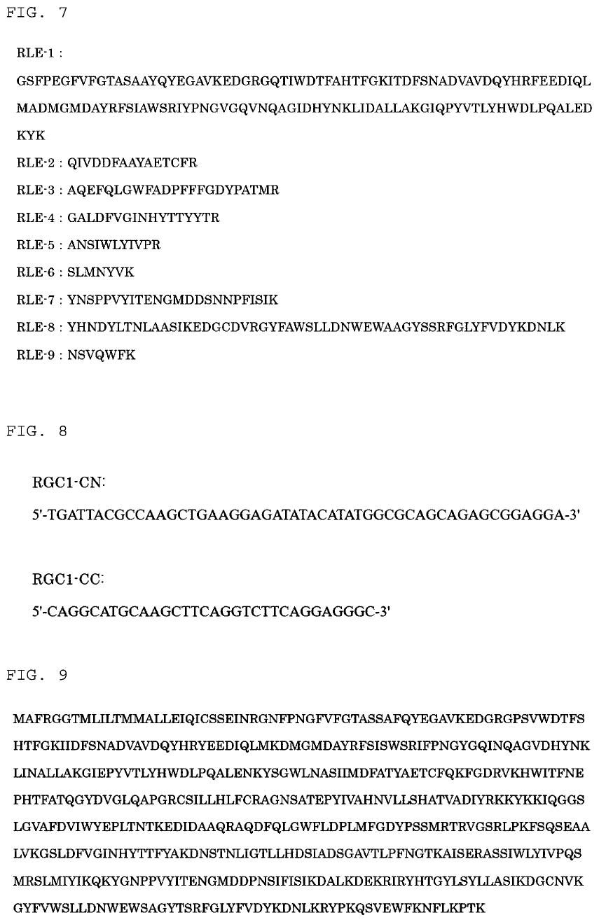

The RGC1 band obtained by SDS-PAGE in Example 2 was excised and then treated with trypsin. The molecular masses of the obtained peptide fragments were analyzed with a matrix assisted laser desorption/ionization time-of-flight mass spectrometer (MALDI-TOFMS: Microflex LRF 20, manufactured by Bruker Daltonics) to determine the amino acid sequences of the 9 peptide fragments shown in SEQ ID NOs: 5 to 13 in the sequence listing and FIG. 7.

The precise molecular weights of these 9 peptide fragments of RGC1 were completely consistent with the precise molecular weights of 9 peptides derived from a rice (Oryza sativa L. cv. Nipponbare)-derived protein called Os3BGlu6 with an unidentified function, suggesting the possibility that RGC1 was a protein identical to Os3BGlu6. It should be noted that there has been no report so far stating that Os3BGlu6 is glucocerebrosidase.

### Example 8

Total RNA was extracted from the callus of rice (Oryza sativa L. cv. Nipponbare) using RNeasy Plant Mini Kit (manufactured by Qiagen N. V., registered trademark (the same holds true for the description below)). mRNA was purified from the total RNA using Absolutely mRNA Purification Kit (manufactured by Agilent Technologies, Inc.). cDNA synthesis from the mRNA and subsequent RACE analysis were performed using SuperScript III Reverse Transcriptase (manufactured by Thermo Fisher Scientific, Inc., registered trademark). Os3BGlu6 cDNA was obtained by PCR amplification using the two primers (F-primer (RGC1-CN), and R-primer (RGC1-CC)) shown in SEQ ID NOs: 14 and 15 in the sequence listing and FIG. 8 which were prepared from an open reading frame presumed from the whole genomic sequence of rice (Oryza sativa L. cv. Nipponbare).

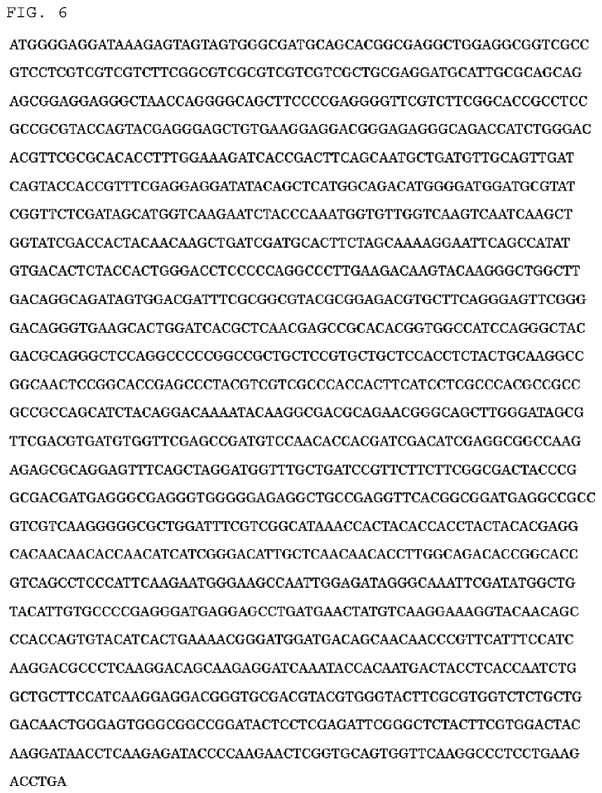

Specific conditions for PCR involved adding KOD-Plus-Neo (manufactured by Toyobo Co., Ltd.) to the rice callus cDNA and the two primers, and repeating reactions conditions of 94° C. for 2 minutes, 60° C. for 0.5 minutes, and 68° C. for 1 minute 35 times for amplification. Then, the amplified fragment was subcloned into a plasmid vector pUC19. Further, a longer DNA region also including DNA regions upstream and downstream of the amplified fragment was amplified by a similar method, and the nucleotide sequence of the fragment was analyzed by a standard method to thereby determine the whole nucleotide sequence of cDNA of the Os3BGlu6 gene. This nucleotide sequence is shown in SEQ ID NO: 3 in the sequence listing and FIG. 6. An amino acid sequence translated from this nucleotide sequence is shown in SEQ ID NO: 1 in the sequence listing and FIG. 5. This amino acid sequence revealed that Os3BGlu6 belonged to GH1.

### Example 9

The presence or absence of glucocerebrosidase activity in Os3BGlu6 produced in Escherichia coli was examined in order to reveal that the isolated Os3BGlu6 gene was glucocerebrosidase gene (gene encoding RGC1). pUC19 obtained in Example 8 in which the Os3BGlu6 gene was subcloned was used. Escherichia coli (DH5a) was transformed by transfer thereof. Next, this transformant was cultured at 37° C. for 24 hours in LB liquid medium (1.0% tryptone, 0.5% yeast extracts, 1.0% sodium chloride, 50 μg/mL ampicillin) and centrifuged under a condition of 15000 rpm for 10 minutes to harvest the bacterial cells. The obtained bacterial cells were washed twice with a 50 mM acetate buffer solution (pH 6.0) containing 0.05% sodium cholate. Then, centrifugation was performed under a condition of 15000 rpm for 10 minutes to harvest the bacterial cells. The obtained bacterial cells were suspended in a 50 mM acetate buffer solution (pH 6.0) containing 0.3% Triton X-100 and 0.05% sodium cholate, followed by sonication. This sonicated homogenate was centrifuged for 60 minutes under conditions of 18000 rpm and 4° C. Then, the centrifuged supernatant was filtered through Durapore Membrane Filter 0.45 μm HV (manufactured by Merck Millipore, registered trademark (the same holds true for the description below)), and then desalted and concentrated to obtain an enzyme extract. This enzyme extract was added to a 50 mM sodium acetate buffer solution (pH 5.5) containing 100 μM glucosylceramide (cerebroside C), 0.1% Tween 20, and 0.05% sodium cholate, and incubated at 37° C. for 13 minutes. Subsequently, a 4-fold amount of ethanol was added to the obtained reaction solution, mixed therewith, and then centrifuged under a condition of 15000 rpm for 20 minutes. The supernatant was subjected to high-performance liquid chromatography analysis to measure the amount of ceramide generated through enzymatic reaction. The high-performance liquid chromatography analysis was carried out in the same manner as in Example 1. The molecular weight of the generated ceramide was examined by negative LC-MS (manufactured by Agilent Technologies, Inc.) to thereby confirm that it was ceramide generated from cerebroside C (ESI-MS m/z: 590.5[M-H]⁻). Glucocerebrosidase activity per mL of the enzyme solution was calculated when the amount of the enzyme that generated 1 μmol of ceramide for 1 minute in the enzymatic reaction solution was defined as 1 unit (U). The results are shown in Table 5 below.

TABLE 5

| | Glucocerebrosidase |
|---|---|
| | activity (U/mL) |
| Escherichia coli transformed | 0.180 |
| with Os3BGlu6 gene | |
| Escherichia coli transformed | 0.000 |
| with only plasmid | |

The results indicated that the enzyme extract obtained from Escherichia coli transformed with the Os3BGlu6 gene evidently had glucocerebrosidase activity. This revealed that the isolated RGC1 was Os3BGlu6 and was glucocerebrosidase belonging to GH1. It should be noted that there has been no report so far stating that glucocerebrosidase belonging to GH1 has been found from plants.

### Example 10

The homology (identity) between the amino acid sequence of RGC1 and the amino acid sequences of animal-derived glucocerebrosidase or thale-cress-derived glucocerebrosidase AtGCD3 was examined using EMBOSS Needle (https://www.ebi.ac.uk/Tools/psa/emboss needle/). As a result, the homology was 14.9% to GBA1, 10.1% to GBA2, 35.8% to GBA3, 11.3% to lactase-phlorizin hydrolase, and 3.3% to AtGCD3. This result revealed that RGC1 was glucocerebrosidase that was also totally different in amino acid sequence from the previously isolated glucocerebrosidase.

### Example 11

102 g of stems of soybean (Glycine max (L.) Merr. Enrei) was collected, chopped, and then homogenized with a homogenizer in a 20 mM acetate buffer solution (pH 5.5) containing 0.01% sodium cholate. This homogenate was centrifuged for 60 minutes under conditions of 10000 rpm and 4° C. The centrifuged supernatant was removed. Further, a 20 mM acetate buffer solution (pH 5.5) containing 0.01% sodium cholate was added to the centrifuged precipitate, stirred for 5 minutes, and then centrifuged for 60 minutes under conditions of 10000 rpm and 4° C. The centrifuged supernatant was removed. Further, an enzyme extract was obtained from the centrifuged precipitate in the same manner as in Example 2.

This enzyme extract was applied to HiTrap Q HP column (manufactured by Amersham Biosciences Corp.) equilibrated with a 0.5 mM phosphate buffer solution (pH 6.7) containing 0.05% sodium cholate and 0.05% Triton X-100. Then, elution was performed by the gradient elution method from the 0.5 mM phosphate buffer solution (pH 6.7) containing 0.05% sodium cholate and 0.05% Triton X-100 into a solution containing 1 M sodium chloride in a 20 mM phosphate buffer solution (pH 6.7) containing 0.05% sodium cholate and 0.1% Triton X-100. Next, a fraction with strongly observed glucocerebrosidase activity was pooled, and desalted and concentrated by ultrafiltration. This desalted and concentrated solution was applied to HiTrap SP HP column (manufactured by Amersham Biosciences Corp.) equilibrated with a 0.5 mM acetate buffer solution (pH 6.0) containing 0.05% sodium cholate and 0.05% Triton X-100. Then, elution was performed for fractionation by the gradient elution method from the 0.5 mM acetate buffer solution (pH 6.0) containing 0.05% sodium cholate and 0.05% Triton X-100 into a solution containing 1 M sodium chloride in a 0.5 mM acetate buffer solution (pH 6.0) containing 0.05% sodium cholate and 0.05% Triton X-100. As a result, a fraction with strongly observed glucocerebrosidase activity was pooled, and desalted and concentrated by ultrafiltration. The resulting solution was used as soybean-derived glucocerebrosidase (SGC1).

The glucocerebrosidase activity of the fraction and the protein concentration of SGC1 were analyzed by the same methods as in Example 2. This SGC1 fraction exhibited a single band in SDS-PAGE, and its average molecular weight (MW) was approximately 62.0 kD. The SDS-PAGE was also performed by the same method as in Example 2.

### Example 12

The optimum temperatures for the glucocerebrosidase activity of SGC1 obtained in Example 11 and imiglucerase were confirmed by the same method as in Example 5. The results are shown in Table 6 below.

From the results, the optimum temperature of imiglucerase was 42.5° C., whereas the optimum temperature of SGC1 was very high, namely 64.0° C., suggesting the possibility that SGC1 was highly stable in vivo.

TABLE 6

| | Optimum |
|---|---|
| | temperature (° C.) |
| Imiglucerase | 42.5 |
| SGC1 | 64.0 |

### Example 13

For enzymes, as mentioned above, it is known that a higher optimum temperature leads to better thermostability. From Example 12, SGC1 was expected to have thermostability because its optimum temperature for glucocerebrosidase activity was higher than that of imiglucerase. Accordingly, the following test was carried out in order to confirm thermostability in lysosome (pH 5) and cytoplasm (pH 7).

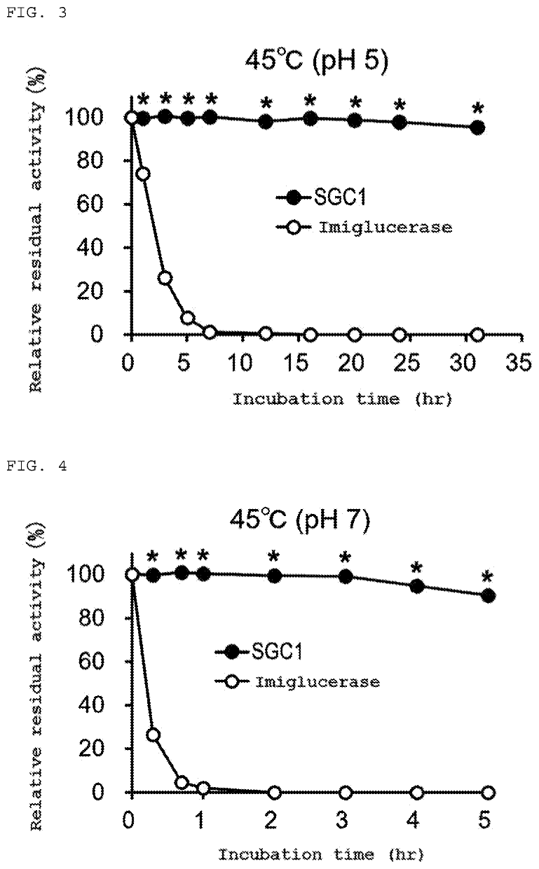

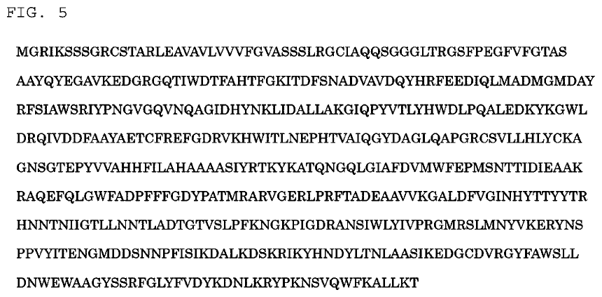

Two glucocerebrosidases, SGC1 obtained in Example 11 and imiglucerase, were each incubated at pH 5 simulating lysosome (50 mM acetate buffer solution (pH 5.0) containing 0.1% Triton X-100 and 0.05% sodium cholate) at 45° C. for 1 to 31 hours or at pH 7 simulating cytoplasm (50 mM potassium phosphate buffer solution (pH 7.0) containing 0.1% Triton X-100 and 0.05% sodium cholate) at 45° C. for 0.3 to 5 hours. Glucocerebrosidase activity after the reaction for each time was measured in accordance with the method of Example 2. Residual activity was calculated as a relative activity value when activity before incubation was defined as 100. This test was conducted three times, and an average value was determined therefrom. The results are shown in FIGS. 3 and 4. In these figures, the mark * was added when the glucocerebrosidase activity of SGC1 thus reacted for each time was significantly higher than that of imiglucerase (1% significance).

From FIGS. 3 and 4, SGC1 was evidently much superior in stability to imiglucerase at both pH 5 and pH 7, indicating that it had thermostability.

### Example 14

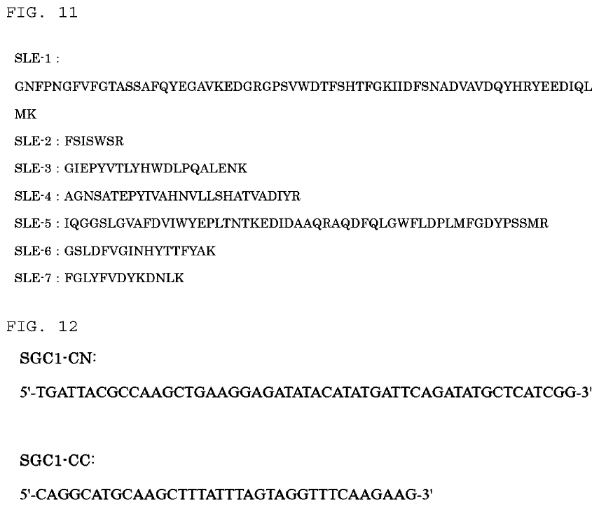

The SGC1 band obtained by SDS-PAGE in Example 11 was excised and then treated with trypsin. The molecular masses of the obtained peptide fragments were analyzed with a matrix assisted laser desorption/ionization time-of-flight mass spectrometer (MALDI-TOFMS: Microflex LRF 20, manufactured by Bruker Daltonics) to determine the amino acid sequences of the 7 peptide fragments shown in SEQ ID NOs: 16 to 22 in the sequence listing and FIG. 11.

The precise molecular weights of these 7 peptide fragments derived from SGC1 were completely consistent with the precise molecular weights of 7 peptides derived from a U.S. soybean cultivar (Glycine max (L.) Merr. Williams 82)-derived protein called β-glucosidase 40 with an unidentified function, suggesting the possibility that SGC1 was a protein identical to β-glucosidase 40. It should be noted that this β-glucosidase 40 has been simply designated from homology search with genomic sequences and has not been confirmed to have enzymatic activity as β-glucosidase; and furthermore, there has been no report so far stating that it is glucocerebrosidase.

### Example 15

Total RNA was extracted from the leaves of soybean (Glycine max (L.) Merr. Enrei) using RNeasy Plant Mini Kit (manufactured by Qiagen N. V.). cDNA was synthesized from this total RNA using PrimeScript II 1st strand cDNA Synthesis Kit (manufactured by Takara Bio Inc.). cDNA of β-glucosidase 40 derived from soybean (Glycine max (L.) Merr. Enrei) was obtained by PCR amplification using the two primers (F-primer (SGC1-CN) and R-primer (SGC1-CC)) shown in SEQ ID NOs: 23 and 24 in the sequence listing and FIG. 12 which were prepared from an open reading frame presumed from the whole genomic sequence of the U.S. soybean cultivar (Glycine max (L.) Merr. Williams 82).

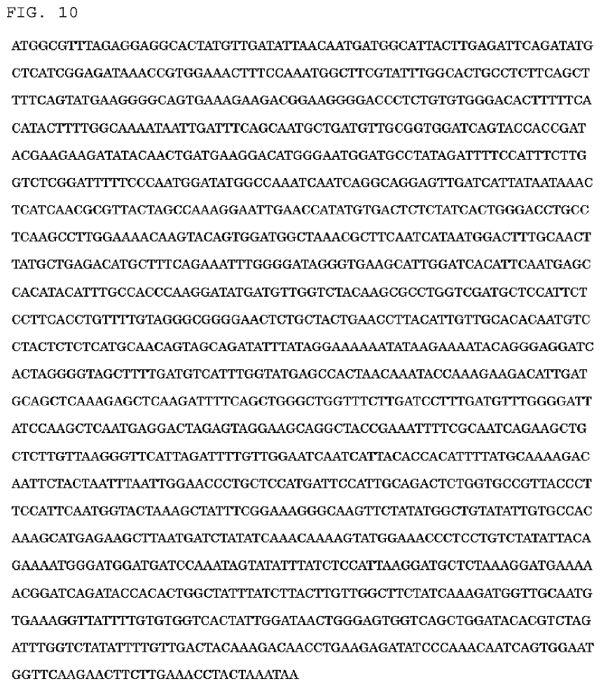

Specific conditions for PCR involved adding KOD-Plus-Neo (manufactured by Toyobo Co., Ltd.) to the cDNA from the leaves of soybean (Glycine max (L.) Merr. Enrei) and the two primers, and repeating reaction conditions of 94° C. for 2 minutes, 60° C. for 0.5 minutes, and 68° C. for 1 minute 40 times for amplification. Then, the amplified fragment was subcloned into a plasmid vector pUC19. Further, a longer DNA region also including DNA regions upstream and downstream of the amplified fragment was amplified by a similar method, and the nucleotide sequence of the fragment was analyzed by a standard method to thereby determine the whole nucleotide sequence of cDNA of the β-glucosidase 40 gene derived from soybean (Glycine max (L.) Merr. Enrei). This nucleotide sequence is shown in SEQ ID NO: 4 in the sequence listing and FIG. 10. An amino acid sequence translated from this nucleotide sequence is shown in SEQ ID NO: 2 in the sequence listing and FIG. 9. This amino acid sequence revealed that β-glucosidase 40 derived from soybean (Glycine max (L.) Merr. Enrei) belonged to GH1.

### Example 16

The presence or absence of glucocerebrosidase activity in soybean-derived β-glucosidase 40 produced in Escherichia coli was examined in order to reveal that the β-glucosidase 40 gene derived from soybean (Glycine max (L.) Merr. Enrei) was glucocerebrosidase gene. pUC19 obtained in Example 15 in which the soybean-derived β-glucosidase 40 gene was subcloned was used. Escherichia coli (DH5a) was transformed by transfer thereof. Glucocerebrosidase activity per mL of an enzyme extract was calculated by the same method as in Example 9 except that the enzymatic reaction was incubated at 60° C. for 30 minutes using this transformant. The results are shown in Table 7 below.

TABLE 7

| | Glucocerebrosidase |
|---|---|
| | activity (U/mL) |
| Escherichia coli transformed with | 0.0123 |
| soybean-derived β-glucosidase 40 | |
| gene | |
| Escherichia coli transformed with | 0.0000 |
| only plasmid | |

The results indicated that the enzyme extract obtained from Escherichia coli transformed with the β-glucosidase 40 gene derived from soybean (Glycine max (L.) Merr. Enrei) evidently had glucocerebrosidase activity. This revealed that the isolated soybean-derived SGC1 was soybean-derived β-glucosidase 40 and was glucocerebrosidase belonging to GH1. As mentioned above, it should be noted that there has been no report so far stating that glucocerebrosidase belonging to GH1 has been found from plants.

### Example 17

The homology (identity) between the amino acid sequence of SGC1 and the amino acid sequences of animal-derived glucocerebrosidase or thale-cress-derived glucocerebrosidase AtGCD3 was examined using EMBOSS Needle described above. As a result, the homology was 14.9% to GBA1, 10.0% to GBA2, 35.9% to GBA3, 10.4% to lactase-phlorizin hydrolase, and 7.6% to AtGCD3. This result revealed that SGC1 was glucocerebrosidase that was also totally different in amino acid sequence from the previously isolated glucocerebrosidase.

### Example 18

A protein having high homology to the amino acid sequence of RGC1 was searched for by use of BLASTp search (https://blast.ncbi.nlm.nih.gov/Blast.cgi). As a result, it was revealed that a wide range of seed plants had highly homologous proteins. The homology (identity) was examined using EMBOSS Needle described above. The results are shown in Table 8 below.

TABLE 8

| | | Homology |
|---|---|---|
| Accession No. and protein name | Origin | (%) |
| Monocotyledonous plant | | |
| XP_015613193.1 beta-glucosidase 34 | Rice (Oryza sativa Japonica Group) | 77.4% |
| VAI08420.1 unnamed protein product | Wheat (Triticum turgidum subsp. durum) | 84.4% |
| KAE8788864.1 Beta-glucosidase 34 | Barley (Hordeum vulgare) | 73.8% |
| PWZ58470.1 Beta-glucosidase 6 | Maize (Zea mays) | 84.2% |
| XP_002465652.2 beta-glucosidase 6 | Sorghum (Sorghum bicolor) | 82.7% |
| RLN17293.1 beta-glucosidase 6 | Proso millet (Panicum miliaceum) | 83.7% |
| XP_004985215.1 beta-glucosidase 6 | Foxtail millet (Setaria italica) | 84.9% |
| Dicotyledonous plant | | |
| NP_173978.1 beta glucosidase 40 | Arabidopsis thaliana | 68.8% |
| XP_013642074.1 beta-glucosidase 40-like | Rapeseed (Brassica napus) | 69.5% |
| XP_013587431.1 beta-glucosidase 40 | Cabbage (Brassica oleracea var. oleracea) | 69.5% |
| XP_011657400.1 PREDICTED: beta-glucosidase 40 | Cucumber (Cucumis sativus) | 68.1% |
| XP_008445465.1 PREDICTED: beta-glucosidase 40 | Melon (Cucumis melo) | 67.9% |
| XP_004228406.1 beta-glucosidase 40 | Tomato (Solanum lycopersicum) | 63.6% |
| XP_016497770.1 beta-glucosidase 40-like | Tobacco (Nicotiana tabacum) | 64.6% |
| XP_006365136.1 beta-glucosidase 40-like | Potato (Solanum tuberosum) | 64.2% |
| XP_003556662.1 beta-glucosidase 40 | Soybean (Glycine max (L.) Merr. Enrei) | 67.6% |
| | SGC1 | |
| XP_019445904.1 beta-glucosidase 40-like | Lupine (Lupinus angustifolius) | 67.0% |
| PRQ40673.1 putative beta-glucosidase | Rosa chinensis | 62.6% |
| PQQ20207.1 beta-glucosidase 40 | Cherry (Prunus yedoensis var. nudiflora) | 65.0% |
| VVA37410.1 PREDICTED: beta-glucosidase | Almond (Prunus dulcis) | 69.4% |
| XP_023754232.1 beta-glucosidase 40 | Lettuce (Lactuca sativa) | 67.0% |
| XP_017616432.1 PREDICTED: beta-glucosidase 40 | Cotton plant (Gossypium arboreum) | 68.8% |
| KAE8732915.1 Beta-glucosidase 34 | Hibiscus (Hibiscus syriacus) | 65.8% |
| XP_017224511.1 beta-glucosidase 6-like | Carrot (Daucus carota subsp. sativus) | 70.0% |
| Fern | | |
| XP_002967091.1 beta-glucosidase 40 | Selaginella moellendorffii | 53.2% |
| Liverwort | | |
| PTQ42983.1 hypothetical protein | Marchantia polymorpha | 46.6% |
| XP_024362173.1 beta-glucosidase 6-like | Physcomitrella patens | 51.4% |

From Table 8, the amino acid sequence of RGC1 exhibited homology of 73.8 to 84.9% to β-glucosidase 6 and β-glucosidase 34 derived from monocotyledonous plants, and exhibited homology of 62.6 to 70.0% to β-glucosidase 6, β-glucosidase 40 and β-glucosidase 34 derived from dicotyledonous plants. It also exhibited homology of 46.6 to 53.2% to fern or liverwort β-glucosidase 6 or β-glucosidase 40. All of these amino acid sequences had a conservative region necessary for GH1 and belonged to GH1. From this result, the β-glucosidase 6, the β-glucosidase 40 and the β-glucosidase 34 described above were considered glucocerebrosidase.

### Example 19

The following test was carried out in order to examine the presence or absence of similar glucocerebrosidase activity in plants other than rice. 0.01 to 1 g each of sites of leaves, roots, stems, petals, pistils, pollens, fruits, rhizoids, and sporangia of various plants was collected and homogenized with a homogenizer in a 50 mM acetate buffer solution (pH 5.0) containing 0.05% sodium cholate and 0.3% Triton X-100. This homogenate was centrifuged for 20 minutes under conditions of 15000 rpm and 4° C. The centrifuged supernatant was used as an enzyme extract.

Then, this enzyme extract was incubated at 45° C. for 60 minutes in a 50 mM acetate buffer solution (pH 5.0) containing 100 μM glucosylceramide (cerebroside B), 0.05% sodium cholate, and 0.2% Triton X-100. Subsequently, a 4-fold amount of ethanol was added to the obtained reaction solution, mixed therewith, and then centrifuged under a condition of 15000 rpm for 20 minutes. The supernatant was subjected to high-performance liquid chromatography analysis to measure the amount of ceramide generated through enzymatic reaction. The high-performance liquid chromatography analysis was carried out in the same manner as in Example 2. The amount of ceramide and enzymatic activity were also calculated in the same manner as in Example 2. The number of units of glucocerebrosidase activity per g of a plant raw weight was calculated. This test was conducted three times, and an average value was determined therefrom. The results are shown in Table 9 below.

TABLE 9

| | Glucocerebrosidase activity (U/g): — represents unmeasured | | | | | | | | |
|---|---|---|---|---|---|---|---|---|---|
| | Leaf | Stem | Root | Petal | Pistil | Pollen | Fruit | Rhizoid | Sporangium |
| Wheat | 0.000486 | 0.000985 | 0.000363 | — | 0.0196 | 0.12 | — | — | — |
| Rice | 0.0092 | 0.0196 | 0.057 | — | 0.07 | 0.94 | 0.0138 | — | — |
| Maize | 0.00105 | 0.0013 | 0.00172 | — | 0.00339 | 0.4 | 0.00344 | — | — |
| Tulip | 0.00031 | 0.00446 | 0.00186 | 0.00218 | 0.0104 | 0.253 | — | — | — |
| Lily | 0.00617 | 0.00724 | 0.00493 | 0.00226 | 0.00498 | 0.0389 | — | — | — |
| Goldenmane tickseed | 0.00189 | 0.00139 | 0.000966 | 0.027 | 0.00335 | 0.212 | — | — | — |
| Gazania | 0.000128 | 0.000159 | 0.000343 | 0.0113 | 0.0381 | 0.0443 | — | — | — |
| Gerbera | 0.000167 | 0.000135 | 0.000209 | 0.000755 | 0.0343 | 0.0112 | — | — | — |
| Rapeseed | 0.0134 | 0.0042 | 0.000489 | 0.0095 | 0.0284 | 0.33 | — | — | — |
| Arabidopsis thaliana | 0.0172 | 0.0422 | 0.000366 | 0.0109 | 0.0364 | 0.242 | 0.0185 | — | — |
| Cabbage | 0.0153 | 0.00898 | 0.000912 | 0.00471 | 0.025 | 0.183 | | — | — |
| Melon | 0.00527 | 0.00278 | 0.00165 | 0.000335 | 0.000457 | 0.26 | 0.000022 | — | — |
| Cucumber | 0.000622 | 0.00161 | 0.00123 | 0.00116 | 0.00363 | 0.504 | 0.000547 | — | — |
| Broad bean | 0.0224 | 0.0142 | 0.00431 | 0.00671 | 0.00864 | 0.18 | 0.00621 | — | — |
| Soybean | 0.0111 | 0.0175 | 0.00516 | 0.00163 | 0.00265 | 0.117 | 0.00162 | — | — |
| Cotton plant | 0.00134 | 0.000548 | 0.00228 | 0.00313 | 0.000179 | 0.0237 | — | — | — |
| Hibiscus | 0.0143 | 0.00853 | 0.00366 | 0.00174 | 0.00208 | 0.105 | — | — | — |
| Cherry | 0.000028 | 0.000029 | 0.000157 | 0.000545 | 0.00021 | 0.152 | — | — | — |
| Rose | 0.000023 | 0.000096 | 0.000354 | 0.000663 | 0.0105 | 0.013 | — | — | — |
| Eggplant | 0.00233 | 0.00531 | 0.00107 | 0.0028 | 0.0193 | 0.74 | 0.00161 | — | — |
| Tomato | 0.000435 | 0.000106 | 0.000416 | 0.000819 | 0.00488 | 0.816 | 0.00128 | — | — |
| Tobacco | 0.00555 | 0.00191 | 0.00171 | 0.000973 | 0.0264 | 0.197 | — | — | — |
| Carrot | 0.002 | 0.00316 | 0.00183 | 0.0119 | 0.0154 | 0.449 | — | — | — |
| Japanese iris | 0.00314 | 0.00471 | 0.00342 | 0.00866 | 0.00366 | 0.0377 | — | — | — |
| Gingko | 0.000744 | 0.0198 | 0.00113 | — | 0.0000217 | 0.0296 | 0.000005 | — | — |
| Pine | 0.000772 | 0.00231 | 0.000368 | — | 0.000013 | 0.042 | — | — | — |
| Cryptomeria japonica | 0.0000953 | 0.0135 | 0.000399 | — | 0.0000403 | 0.46 | — | — | — |
| Thelypteris | 0.0000337 | 0.00000933 | 0.000013 | — | — | — | — | — | 0.000215 |
| acuminata | | | | | | | | | |
| Dryopteris | 0.000432 | 0.000194 | 0.00052 | — | — | — | — | — | 0.000299 |
| crassirhizoma | | | | | | | | | |
| Selaginella | 0.0000117 | 0.000091 | 0.000359 | — | — | — | — | — | 0.0000913 |
| moellendorffii | | | | | | | | | |
| Equisetum arvense | 0.0000857 | 0.000219 | 0.000294 | — | — | — | — | — | 0.00056 |
| Lygodium japonicum | 0.000013 | 0.000163 | 0.000016 | — | — | — | — | — | 0.000008 |
| Pogonatum inflexum | 0.000123 | 0.000408 | — | — | — | — | — | 0.000149 | — |
| Pseudospiridentopsis | 0.000194 | 0.000334 | — | — | — | — | — | 0.000608 | — |
| horrida | | | | | | | | | |
| Hypnum | 0.000068 | 0.0000297 | — | — | — | — | — | 0.000186 | — |
| plumaeforme | | | | | | | | | |
| Pyrrhobryum | 0.00000733 | 0.0000563 | — | — | — | — | — | 0.000077 | — |
| dozyanum | | | | | | | | | |
| Campylopus | 0.00000767 | 0.000122 | — | — | — | — | — | 0.0000613 | — |
| umbellatus | | | | | | | | | |
| Dicranum japonicum | 0.000064 | 0.0000467 | — | — | — | — | — | 0.000313 | — |

From the results, glucocerebrosidase activity was observed in the measured extracts of all the plants. In consideration of the results of Tables 8 and 9, glucocerebrosidase was shown to exist in not only rice or soybean but all plants.

Furthermore, seed plants were shown to have glucocerebrosidase belonging to GH1 because monocotyledonous plant-derived RGC1 and dicotyledonous plant-derived SGC1 were glucocerebrosidase belonging to GH1 (Examples 9 and 16); enzymes that had high homology to the amino acid sequence of RGC1 and belonged to GH1 existed in many seed plants (Example 18); and as shown in Table 9 described above, high glucocerebrosidase activity was detected from extracts from many seed plants. Furthermore, this is enough to suggest that fern and liverwort also have glucocerebrosidase belonging to GH1 because β-glucosidase 6 or β-glucosidase 40 existed in fern or liverwort (Table 8 mentioned above); and glucocerebrosidase activity was also detected in extracts from fern or liverwort.

Next, homology will be discussed. Monocotyledonous plant-derived RGC1 and dicotyledonous plant-derived SGC1 demonstrated to be glucocerebrosidase belonging to GH1 in Examples 9 and 16 have homology of 67.6% between their amino acid sequences. It is therefore considered that an enzyme at least having homology equal to or more than 67.5% to the amino acid sequences of RGC1 and SGC1 is reliably glucocerebrosidase belonging to GH1. From Table 9 described above, seed plants were found to have glucocerebrosidase. It is therefore considered that an enzyme at least having homology equal to or more than 62.6% (China rose) which is the lowest homology to the amino acid sequence of RGC1 among the seed plants as shown in Table 8 mentioned above is also glucocerebrosidase belonging to GH1.

### Example 20

Enzyme extracts derived from various plants which were obtained in accordance with Example 19, the purified rice-derived RGC1 mentioned above and the purified soybean-derived SGC1 mentioned above were each added to a 50 mM sodium acetate buffer solution (pH 5.5) containing 100 μM glucosylceramide (cerebroside B), 0.1% Tween 20, and 0.05% sodium cholate, and incubated at each temperature for 15 minutes. Subsequently, a 4-fold amount of ethanol was added to the obtained reaction solution, mixed therewith, and then centrifuged at 15000 rpm for 20 minutes. The supernatant was subjected to high-performance liquid chromatography analysis to measure the amount of ceramide generated through enzymatic reaction. The high-performance liquid chromatography analysis was carried out in the same manner as in Example 5. The amount of ceramide and enzymatic activity were also calculated in the same manner as in Example 5. As a result, a temperature that attained the highest glucocerebrosidase activity was regarded as the optimum temperature. This test was conducted three times, and an average value was determined therefrom. The results are shown in Table 10 below.

TABLE 10

| | Optimum |
|---|---|
| | temperature (° C.) |
| Human-derived glucocerebrosidase (imiglucerase) | 42.5 |
| Rice-derived glucocerebrosidase (RGC1) | 54.0 |
| Maize-derived glucocerebrosidase | 55.5 |
| Wheat-derived glucocerebrosidase | 51.6 |
| Tulip-derived glucocerebrosidase | 47.7 |
| Narcissus-derived glucocerebrosidase | 48.7 |
| Morning star lily-derived glucocerebrosidase | 54.4 |
| Arabidopsis thaliana-derived glucocerebrosidase | 53.9 |
| Cabbage-derived glucocerebrosidase | 56.3 |
| Japanese radish-derived glucocerebrosidase | 54.2 |
| Rapeseed-derived glucocerebrosidase | 53.1 |
| Bitter cucumber-derived glucocerebrosidase | 60.4 |
| Melon-derived glucocerebrosidase | 63.4 |
| Cucumber-derived glucocerebrosidase | 64.0 |
| Lettuce-derived glucocerebrosidase | 54.6 |
| Gerbera-derived glucocerebrosidase | 62.6 |
| Gazania-derived glucocerebrosidase | 57.5 |
| Goldenmane tickseed-derived glucocerebrosidase | 57.5 |
| Tomato-derived glucocerebrosidase | 54.2 |
| Eggplant-derived glucocerebrosidase | 53.7 |
| Tobacco-derived glucocerebrosidase | 56.8 |
| Cherry-derived glucocerebrosidase | 56.6 |
| Rose-derived glucocerebrosidase | 61.7 |
| Lupine-derived glucocerebrosidase | 50.6 |
| Japanese wisteria-derived glucocerebrosidase | 56.8 |
| Broad bean-derived glucocerebrosidase | 62.5 |
| Soybean-derived glucocerebrosidase (SGC1) | 64.0 |

From the results, glucocerebrosidase derived from many seed plants including rice and soybean was also found to have a higher optimum temperature than that of imiglucerase, human-derived glucocerebrosidase. As shown in Examples 6 and 13, for enzymes, it is generally known that a higher optimum temperature leads to better thermostability. Therefore, it can be concluded that seed plant-derived glucocerebrosidase belonging to GH1 is superior in stability against heat to human-derived glucocerebrosidase imiglucerase, that is, has thermostability.

This application claims the priority based on Japanese Patent Application No. 2020-88950 filed on May 21, 2020, the disclosure of which is incorporated herein in its entirety.

1. A protein which is derived from a plant, belongs to glycoside hydrolase family 1, and has glucocerebrosidase activity.

2. The protein according to claim 1, wherein the plant is a seed plant.

3. The protein according to claim 2, wherein the seed plant is one of a plant of the family Brassicaceae, a plant of the family Poaceae, a plant of the family Cucurbitaceae, a plant of the family Compositae, a plant of the family Solanaceae, a plant of the family Rosaceae, a plant of the family Amaryllidaceae, a plant of the family Leguminosae, and a plant of the family Liliaceae.

4. A protein shown in the following (A), (B), or (C):
(A) a protein consisting of an amino acid sequence represented by SEQ ID NO: 1, amino acid positions 38 to 521 of SEQ ID NO: 1, SEQ ID NO: 2, or amino acid positions 19 to 503 of SEQ ID NO: 2 in the sequence listing;
(B) a protein which consists of the amino acid sequence represented by SEQ ID NO: 1, amino acid positions 38 to 521 of SEQ ID NO: 1, SEQ ID NO: 2, or amino acid positions 19 to 503 of SEQ ID NO: 2 in the sequence listing, having one or several nucleotides substituted, deleted, inserted, or added, belongs to glycoside hydrolase family 1, and has glucocerebrosidase activity and thermostability; or
© a protein which has homology equal to or more than 60% to the amino acid sequence represented by SEQ ID NO: 1, amino acid positions 38 to 521 of SEQ ID NO: 1, SEQ ID NO: 2, or amino acid positions 19 to 503 of SEQ ID NO: 2 in the sequence listing, belongs to glycoside hydrolase family 1, and has glucocerebrosidase activity and thermostability.

5. (canceled)

6. DNA shown in the following (a), (b), or (c):
(a) DNA which consists of a nucleotide sequence represented by SEQ ID NO: 3, nucleotide positions 112 to 1566 of SEQ ID NO: 3, SEQ ID NO: 4, or nucleotide positions 55 to 1512 of SEQ ID NO: 4 in the sequence listing, and encodes a protein belonging to glycoside hydrolase family 1 and having glucocerebrosidase activity;
(b) DNA which consists of the nucleotide sequence represented by SEQ ID NO: 3, nucleotide positions 112 to 1566 of SEQ ID NO: 3, SEQ ID NO: 4, or nucleotide positions 55 to 1512 of SEQ ID NO: 4 in the sequence listing, having one or several nucleotides substituted, deleted, inserted, or added, and encodes a protein belonging to glycoside hydrolase family 1 and having glucocerebrosidase activity and thermostability; or
(c) DNA which is capable of hybridizing under stringent conditions to DNA consisting of a nucleotide sequence complementary to the nucleotide sequence represented by SEQ ID NO: 3, nucleotide positions 112 to 1566 of SEQ ID NO: 3, SEQ ID NO: 4, or nucleotide positions 55 to 1512 of SEQ ID NO: 4 in the sequence listing, and encodes a protein belonging to glycoside hydrolase family 1 and having glucocerebrosidase activity and thermostability.

7. An expression vector for the expression of a protein belonging to glycoside hydrolase family 1 and having glucocerebrosidase activity and thermostability, the expression vector comprising DNA according to claim 6.

8. A transformant harboring an expression vector according to claim 7.

9. The transformant according to claim 8, wherein the transformant is one of a plant, a plant cell, an animal cell, Escherichia coli, a yeast, and a filamentous fungus.

10. (canceled)

11. An enzyme composition for glucosylceramide hydrolysis, comprising a protein according to claim 1.

12. A pharmaceutical composition comprising a protein according to claim 1 as an active ingredient.

13. The pharmaceutical composition according to claim 12 for Gaucher's disease prevention and treatment.

14. A food composition comprising a protein according to claim 1, and glucosylceramide isolated from a plant, an animal, or a microbe, or chemically synthesized glucosylceramide.

15. A method for producing ceramide, comprising the step of using a protein according to claim 1 to generate ceramide from glucosylceramide isolated from a plant, an animal, or a microbe, or chemically synthesized glucosylceramide.

16. A method for preventing or treating Gaucher's disease, including administering a protein according to claim 1 to a Gaucher's disease patient.

17. The method for preventing or treating Gaucher's disease according to claim 16, wherein the protein is administered to the Gaucher's disease patient once per 3 to 10 weeks such that glucocerebrosidase activity is 10 to 200 U/kg body weight.


SEQUENCE LISTING (24 sequences)

SEQ ID NO: 1 - protein, 521 aa, Glycine max (L.) Merr. Enrei [sequence omitted]

SEQ ID NO: 2 - protein, 503 aa, Oryza sativa L. cv. Nipponbare
MAFRGGTMLILTMMALLEIQICSSEINRGNFPNGFVFGTASSAFQYEGAVKEDGRGPSVWDTFSHTFGKIIDFSNADVAVDQYHRYEEDIQLMKDMGMDAYRFSISWSRIFPNGYGQINQAGVDHYNKLINALLAKGIEPYVTLYHWDLPQALENKYSGWLNASIIMDFATYAETCFQKFGDRVKHWITFNEPHTFATQGYDVGLQAPGRCSILLHLFCRAGNSATEPYIVAHNVLLSHATVADIYRKKYKKIQGGSLGVAFDVIWYEPLTNTKEDIDAAQRAQDFQLGWFLDPLMFGDYPSSMRTRVGSRLPKFSQSEAALVKGSLDFVGINHYTTFYAKDNSTNLIGTLLHDSIADSGAVTLPFNGTKAISERASSIWLYIVPQSMRSLMIYIKQKYGNPPVYITENGMDDPNSIFISIKDALKDEKRIRYHTGYLSYLLASIKDGCNVKGYFVWSLLDNWEWSAGYTSRFGLYFVDYKDNLKRYPKQSVEWFKNFLKPTK

SEQ ID NO: 3 - DNA, 1566 bp, Glycine max (L.) Merr. Enrei
atggggaggataaagagtagtagtgggcgatgcagcacggcgaggctggaggcggtcgccgtcctcgtcgtcgtcttcggcgtcgcgtcgtcgtcgctgcgaggatgcattgcgcagcagagcggaggagggctaaccaggggcagcttccccgaggggttcgtcttcggcaccgcctccgccgcgtaccagtacgagggagctgtgaaggaggacgggagagggcagaccatctgggacacgttcgcgcacacctttggaaagatcaccgacttcagcaatgctgatgttgcagttgatcagtaccaccgtttcgaggaggatatacagctcatggcagacatggggatggatgcgtatcggttctcgatagcatggtcaagaatctacccaaatggtgttggtcaagtcaatcaagctggtatcgaccactacaacaagctgatcgatgcacttctagcaaaaggaattcagccatatgtgacactctaccactgggacctcccccaggcccttgaagacaagtacaagggctggcttgacaggcagatagtggacgatttcgcggcgtacgcggagacgtgcttcagggagttcggggacagggtgaagcactggatcacgctcaacgagccgcacacggtggccatccagggctacgacgcagggctccaggcccccggccgctgctccgtgctgctccacctctactgcaaggccggcaactccggcaccgagccctacgtcgtcgcccaccacttcatcctcgcccacgccgccgccgccagcatctacaggacaaaatacaaggcgacgcagaacgggcagcttgggatagcgttcgacgtgatgtggttcgagccgatgtccaacaccacgatcgacatcgaggcggccaagagagcgcaggagtttcagctaggatggtttgctgatccgttcttcttcggcgactacccggcgacgatgagggcgagggtgggggagaggctgccgaggttcacggcggatgaggccgccgtcgtcaagggggcgctggatttcgtcggcataaaccactacaccacctactacacgaggcacaacaacaccaacatcatcgggacattgctcaacaacaccttggcagacaccggcaccgtcagcctcccattcaagaatgggaagccaattggagatagggcaaattcgatatggctgtacattgtgccccgagggatgaggagcctgatgaactatgtcaaggaaaggtacaacagcccaccagtgtacatcactgaaaacgggatggatgacagcaacaacccgttcatttccatcaaggacgccctcaaggacagcaagaggatcaaataccacaatgactacctcaccaatctggctgcttccatcaaggaggacgggtgcgacgtacgtgggtacttcgcgtggtctctgctggacaactgggagtgggcggccggatactcctcgagattcgggctctacttcgtggactacaaggataacctcaagagataccccaagaactcggtgcagtggttcaaggccctcctgaagacctga

SEQ ID NO: 4 - DNA, 1512 bp, Glycine max (L.) Merr. Enrei
atggcgtttagaggaggcactatgttgatattaacaatgatggcattacttgagattcagatatgctcatcggagataaaccgtggaaactttccaaatggcttcgtatttggcactgcctcttcagcttttcagtatgaaggggcagtgaaagaagacggaaggggaccctctgtgtgggacactttttcacatacttttggcaaaataattgatttcagcaatgctgatgttgcggtggatcagtaccaccgatacgaagaagatatacaactgatgaaggacatgggaatggatgcctatagattttccatttcttggtctcggatttttcccaatggatatggccaaatcaatcaggcaggagttgatcattataataaactcatcaacgcgttactagccaaaggaattgaaccatatgtgactctctatcactgggacctgcctcaagccttggaaaacaagtacagtggatggctaaacgcttcaatcataatggactttgcaacttatgctgagacatgctttcagaaatttggggatagggtgaagcattggatcacattcaatgagccacatacatttgccacccaaggatatgatgttggtctacaagcgcctggtcgatgctccattctccttcacctgttttgtagggcggggaactctgctactgaaccttacattgttgcacacaatgtcctactctctcatgcaacagtagcagatatttataggaaaaaatataagaaaatacagggaggatcactaggggtagcttttgatgtcatttggtatgagccactaacaaataccaaagaagacattgatgcagctcaaagagctcaagattttcagctgggctggtttcttgatcctttgatgtttggggattatccaagctcaatgaggactagagtaggaagcaggctaccgaaattttcgcaatcagaagctgctcttgttaagggttcattagattttgttggaatcaatcattacaccacattttatgcaaaagacaattctactaatttaattggaaccctgctccatgattccattgcagactctggtgccgttacccttccattcaatggtactaaagctatttcggaaagggcaagttctatatggctgtatattgtgccacaaagcatgagaagcttaatgatctatatcaaacaaaagtatggaaaccctcctgtctatattacagaaaatgggatggatgatccaaatagtatatttatctccattaaggatgctctaaaggatgaaaaacggatcagataccacactggctatttatcttacttgttggcttctatcaaagatggttgcaatgtgaaaggttattttgtgtggtcactattggataactgggagtggtcagctggatacacgtctagatttggtctatattttgttgactacaaagacaacctgaagagatatcccaaacaatcagtggaatggttcaagaacttcttgaaacctactaaataa

SEQ ID NO: 5 - protein, 130 aa, Oryza sativa L. cv. Nipponbare
GSFPEGFVFGTASAAYQYEGAVKEDGRGQTIWDTFAHTFGKITDFSNADVAVDQYHRFEEDIQLMADMGMDAYRFSIAWSRIYPNGVGQVNQAGIDHYNKLIDALLAKGIQPYVTLYHWDLPQALEDKYK

SEQ ID NO: 6 - protein, 15 aa, Oryza sativa L. cv. Nipponbare
QIVDDFAAYAETCFR

SEQ ID NO: 7 - protein, 23 aa, Oryza sativa L. cv. Nipponbare
AQEFQLGWFADPFFFGDYPATMR

SEQ ID NO: 8 - protein, 17 aa, Oryza sativa L. cv. Nipponbare
GALDFVGINHYTTYYTR

SEQ ID NO: 9 - protein, 11 aa, Oryza sativa L. cv. Nipponbare
ANSIWLYIVPR

SEQ ID NO: 10 - protein, 7 aa, Oryza sativa L. cv. Nipponbare
SLMNYVK

SEQ ID NO: 11 - protein, 24 aa, Oryza sativa L. cv. Nipponbare
YNSPPVYITENGMDDSNNPFISIK

SEQ ID NO: 12 - protein, 54 aa, Oryza sativa L. cv. Nipponbare
YHNDYLTNLAASIKEDGCDVRGYFAWSLLDNWEWAAGYSSRFGLYFVDYKDNLK

SEQ ID NO: 13 - protein, 7 aa, Oryza sativa L. cv. Nipponbare
NSVQWFK

SEQ ID NO: 14 - DNA, 51 bp, Artificial sequence
  F-primer for amplifying Oryza sativa L. cv. Nipponbare Os3BGlu6 gene sequence
tgattacgccaagctgaaggagatatacatatggcgcagcagagcggagga

SEQ ID NO: 15 - DNA, 32 bp, Artificial sequence
  R-primer for amplifying Oryza sativa L. cv. Nipponbare Os3BGlu6 gene sequence
caggcatgcaagcttcaggtcttcaggagggc

SEQ ID NO: 16 - protein, 66 aa, Glycine max (L.) Merr. Enrei
GNFPNGFVFGTASSAFQYEGAVKEDGRGPSVWDTFSHTFGKIIDFSNADVAVDQYHRYEEDIQLMK

SEQ ID NO: 17 - protein, 7 aa, Glycine max (L.) Merr. Enrei
FSISWSR

SEQ ID NO: 18 - protein, 20 aa, Glycine max(L.) Merr. Enrei
GIEPYVTLYHWDLPQALENK

SEQ ID NO: 19 - protein, 27 aa, Glycine max (L.) Merr. Enrei
AGNSATEPYIVAHNVLLSHATVADIYR

SEQ ID NO: 20 - protein, 53 aa, Glycine max (L.) Merr. Enrei
IQGGSLGVAFDVIWYEPLTNTKEDIDAAQRAQDFQLGWFLDPLMFGDYPSSMR

SEQ ID NO: 21 - protein, 17 aa, Glycine max (L.) Merr. Enrei
GSLDFVGINHYTTFYAK

SEQ ID NO: 22 - protein, 13 aa, Glycine max (L.) Merr. Enrei
FGLYFVDYKDNLK

SEQ ID NO: 23 - DNA, 52 bp, Artificial sequence
  F-primer for amplifying Glycine max (L.) Merr. Enrei beta-glucosidase 40 gene sequence
tgattacgccaagctgaaggagatatacatatgattcagatatgctcatcgg

SEQ ID NO: 24 - DNA, 36 bp, Artificial sequence
  R-primer for amplifying Glycine max (L.) Merr. Enrei beta-glucosidase 40 gene sequence
caggcatgcaagctttatttagtaggtttcaagaag